SEQUENCE LISTING (52 sequences)

SEQ ID NO: 1 - DNA, 2214 bp, Equus caballus
atgcctctaaaccgcactttgtccatgtcctcactgccaggactggacgattgggaggatgaattcgacctggagaatacagtgctcttcgaggtggcctgggaggtggccaacaaggtgggtggcatctacacggtgctacagacgaaggcgaaggtgacaggggatgaatggggcgacaactactacctggtgggaccatacacggagcaaggcgtgaggacccaggtggagctgctcgagcccccaaccccggccctgaagaggacgctggactccatgaacagcaagggctgcaaggtgtatttcgggcgctggctgatcgaggggggccccctggtggtgctcctggatgtgggggcctcagcctgggccctggaacgctggaagggagagctttgggacacctgcaacatcggggtgccctggtacgaccgtgaggccaacgacgccgtcctttttggcttcctcaccacctggttcctgggtgagttcctggcccagagcgaggagaagccacatgtggttgcacacttccacgagtggttggcgggcatcgggctctgcctgtgccgtgcccggcggctgcctgtggctacaatcttcaccacccacgccacgctgctggggcgatacctgtgtgccggtgctgtggacttctacaacaacctggagaatttcaacgtggacaaggaagctggtgagaggcaaatttatcaccgttactgcatggagcgggcggcagcccactgcactcacgtcttcactaccgtgtcccagatcaccgccattgaggctcagcacctactcaagaggaaaccagatatcgtgacccccaatggactgaatgtgaagaagttctctgccatgcatgagttccagaacctccatgctcagagcaaggcccgaatccaggagtttgtgcgtggccatttttatgggcacctggacttcaacttggataagaccctgtatttctttatcgccggccgctacgagttctccaacaagggggctgacgtcttcctggaggccttggcccggctcaactatctgctcagagtaaatggcagcgagcagacggtggtcgccttcttcatcatgccggctcggaccaacaacttcaacgtggaaaccctcaaggggcaagccgtgcgcaagcagctctgggatacggcgaacacagtgaaggagaagttcgggaggaagctttacgaatccctgctggttgggagcctcccggacatgaacaagatgctggacaaggaggatttcactatgatgaagagagccatctttgccacgcagcggcagtcttttccccctgtgtgcacccacaatatgctggacgactcctcggaccctatcctgaccaccatccgtcgaatcggcctcttcaatagtagtgctgacagggtcaaggtgattttccacccagagttcctctcctccacgagccccctgctccccgtggactatgaggagtttgtccgtggctgccaccttggggttttcccctcctactatgagccttggggctacacaccagctgagtgcacggttatgggcatccccagtatctccaccaacctctccggcttcggctgcttcatggaggaacacatcgcagacccctcagcttacggcatctacattctggaccggcggttccgcagcctggatgattcctgctcgcagcttacctccttcctctacagcttctgccagcagagccggcggcagcgcatcatccagcggaaccgcacggagcgcctctccgaccttctggactggaaatacctaggccggtactatatgtccgcgcgccacatggcgctggccaaggcctttccagaacatttcacctacgagccccgcgaggctgatgcgacccagggctaccgctacccacggcctgcatcggtgcctccgtcgccctcactgtcacgacactcgagcccgcaccagagcgaggacgaggaggagccccgggacgtgccgcccgatgaagacagtgagcgctacgacgaggacgaggaggccgccaaggaccggcgcaacatccgcgccccggagtggccgcgtcgcgcctcctgcacctcttccacgagcgggagcaagcgcggctcggtggacacggggccctccagctcgctcagcacccccagcgagcccctcagccccgccagctccctgggcgaggagcgcaactaa

SEQ ID NO: 2 - DNA, 231 bp, Equus caballus
ttgaaacatggggccttctcccccatgcctagatatcgtgacccccaatggactgaatgtgaagaagttctctgccatgcatgagttccagaacctccatgctcagagcaaggcccgaatccaggagtttgtgcgtggccatttttatgggtatgtgggccagatacccaggtcttgagagaggtgggggttgggtgcccagactcccgggtctaagggggggacagctaa

SEQ ID NO: 3 - DNA, 231 bp, Equus caballus
ttgaaacatggggccttctcccccatgcctagatatcgtgacccccaatggactgaatgtgaagaagttctctgccatgcatgagttccagaacctccatgctcagagcaaggcccgaatccaggagtttgtgcatggccatttttatgggtatgtgggccagatacccaggtcttgagagaggtgggggttgggtgcccagactcccgggtctaagggggggacagctaa

SEQ ID NO: 4 - protein, 39 aa, Equus caballus
IVTPNGLNVKKFSAMHEFQNLHAQSKRRIQEFVHGHFYG

SEQ ID NO: 5 - protein, 39 aa, Unknown
  Description of Unknown: Human, Control Horse, Chimpanzee, Canine, Bovine, Mouse, Rat, Pig or Zebrafish sequence
IVTPNGLNVKKFSAMHEFQNLHAQSKRRIQEFVRGHFYG

SEQ ID NO: 6 - DNA, 886 bp, Equus caballus
gtggggcataggagggggtcccgagacatgaatgaggtagttgacggtctacgccaaggcccgtaccaattttcttattgtaaagagcagagaactacaactcccagaagaccctaagttgatggcctgcatatgaggctgccgggctgtaccctagaggctaatgggaggtgtagtttctttgacattcatttcagaaaagggaaaattattttattggctaaggtggatattggtcttctgaaagaggatggaggttcactggattttgtttttgagaaaagcagtgttagaaccagttattcttcccaggctatagcatcaattctcaatggatgtgagaactacaactcccagaaggcctctggctggccagctccttaatattcctggaattaccttcatttattcattcaaccacattgatctgaatgaagaagtgaaacatgggaccttctcccccatgcctagatatcgtgacccccaatggactgaatgtgaagaagttctctgccatgcatgagttccagaacctccatgctcagagcaaggcccgaatccaggagtttgtgcgtggccatttttatgggtatgtgggccagatacccaggtcttgagagaggtgggggttgggtgcccagactcccgggtctaagggaggggacagctgggggctcagactcttgagttcctgagcgtcagaacaatggctgagtattgcaggagatccccgtttaaggagaaaagaggcacaagtcccatgagtcccatgatgcctctgtactgtctaagatgttctttgcccctgtggcttctggggtttgtagttttgagccagggctagagggtgaggatcttggcttcaccctgtcttgtggctttttag

SEQ ID NO: 7 - DNA, 20 bp, Artificial Sequence
  Description of Artificial Sequence: Synthetic primer
tgaaacatgggaccttctcc

SEQ ID NO: 8 - DNA, 20 bp, Artificial Sequence
  Description of Artificial Sequence: Synthetic primer
agctgtcccctcccttagac

SEQ ID NO: 9 - protein, 737 aa, Equus caballus
MPLNRTLSMSSLPGLDDWEDEFDLENTVLFEVAWEVANKVGGIYTVLQTKAKVTGDEWGDNYYLVGPYTEQGVRTQVELLEPPTPALKRTLDSMNSKGCKVYFGRWLIEGGPLVVLLDVGASAWALERWKGELWDTCNIGVPWYDREANDAVLFGFLTTWFLGEFLAQSEEKPHVVAHFHEWLAGIGLCLCRARRLPVATIFTTHATLLGRYLCAGAVDFYNNLENFNVDKEAGERQIYHRYCMERAAAHCTHVFTTVSQITAIEAQHLLKRKPDIVTPNGLNVKKFSAMHEFQNLHAQSKARIQEFVRGHFYGHLDFNLDKTLYFFIAGRYEFSNKGADVFLEALARLNYLLRVNGSEQTVVAFFIMPARTNNFNVETLKGQAVRKQLWDTANTVKEKFGRKLYESLLVGSLPDMNKMLDKEDFTMMKRAIFATQRQSFPPVCTHNMLDDSSDPILTTIRRIGLFNSSADRVKVIFHPEFLSSTSPLLPVDYEEFVRGCHLGVFPSYYEPWGYTPAECTVMGIPSISTNLSGFGCFMEEHIADPSAYGIYILDRRFRSLDDSCSQLTSFLYSFCQQSRRQRIIQRNRTERLSDLLDWKYLGRYYMSARHMALAKAFPEHFTYEPREADATQGYRYPRPASVPPSPSLSRHSSPHQSEDEEEPRDVPPDEDSERYDEDEEAAKDRRNIRAPEWPRRASCTSSTSGSKRGSVDTGPSSSLSTPSEPLSPASSLGEERN

SEQ ID NO: 10 - protein, 11 aa, Equus caballus
QEFVRGHFYGH

SEQ ID NO: 11 - DNA, 19 bp, Artificial Sequence
  Description of Artificial Sequence: Synthetic primer
acagagctgagggccaatc

SEQ ID NO: 12 - DNA, 20 bp, Artificial Sequence
  Description of Artificial Sequence: Synthetic primer
ccggctccctgttactcaag

SEQ ID NO: 13 - DNA, 21 bp, Artificial Sequence
  Description of Artificial Sequence: Synthetic primer
aaagatctctgtgctccctca

SEQ ID NO: 14 - DNA, 19 bp, Artificial Sequence
  Description of Artificial Sequence: Synthetic primer
ggccctgcagtgtaacctt

SEQ ID NO: 15 - DNA, 20 bp, Artificial Sequence
  Description of Artificial Sequence: Synthetic primer
gggatgtggctcagagactg

SEQ ID NO: 16 - DNA, 20 bp, Artificial Sequence
  Description of Artificial Sequence: Synthetic primer
tagcggtagccctgggtctg

SEQ ID NO: 17 - DNA, 19 bp, Artificial Sequence
  Description of Artificial Sequence: Synthetic primer
ctccgctcactctgcagtc

SEQ ID NO: 18 - DNA, 20 bp, Artificial Sequence
  Description of Artificial Sequence: Synthetic primer
acctctggcaccacagtagg

SEQ ID NO: 19 - DNA, 20 bp, Artificial Sequence
  Description of Artificial Sequence: Synthetic primer
gcactttgtccatgtcctca

SEQ ID NO: 20 - DNA, 20 bp, Artificial Sequence
  Description of Artificial Sequence: Synthetic primer
tttatgggcacctggacttc

SEQ ID NO: 21 - DNA, 21 bp, Artificial Sequence
  Description of Artificial Sequence: Synthetic primer
gaggctcagcacctactcaag

SEQ ID NO: 22 - DNA, 20 bp, Artificial Sequence
  Description of Artificial Sequence: Synthetic primer
catccccagtatctccacca

SEQ ID NO: 23 - DNA, 20 bp, Artificial Sequence
  Description of Artificial Sequence: Synthetic primer
tatgagccttggggctacac

SEQ ID NO: 24 - DNA, 20 bp, Artificial Sequence
  Description of Artificial Sequence: Synthetic primer
cgatgaagacagtgagcgct

SEQ ID NO: 25 - DNA, 20 bp, Artificial Sequence
  Description of Artificial Sequence: Synthetic primer
tgaaacatgggaccttctcc

SEQ ID NO: 26 - DNA, 20 bp, Artificial Sequence
  Description of Artificial Sequence: Synthetic primer
agctgtcccctcccttagac

SEQ ID NO: 27 - protein, 50 aa, Equus caballus
MHEFQNLHAQSKARIQEFVRGHFYGHLDFNLDKTLYFFIAGRYEFSNKGA

SEQ ID NO: 28 - protein, 50 aa, Equus caballus
MHEFQNLHAQSKARIQEFVHGHFYGHLDFNLDKTLYFFIAGRYEFSNKGA

SEQ ID NO: 29 - protein, 50 aa, Homo sapiens; identical to SEQ ID NO: 27

SEQ ID NO: 30 - protein, 50 aa, Macaca mulatta; identical to SEQ ID NO: 27

SEQ ID NO: 31 - protein, 50 aa, Mus musculus; identical to SEQ ID NO: 27

SEQ ID NO: 32 - protein, 50 aa, Oryctolagus cuniculus; identical to SEQ ID NO: 27

SEQ ID NO: 33 - protein, 50 aa, Ornithorhynchus anatinus
MHEFQNLHAQSKARIQEFVRGHFYGHLDFSLDKTLFFFIAGRYEFSNKGA

SEQ ID NO: 34 - protein, 50 aa, Monodelphis domestica; identical to SEQ ID NO: 27

SEQ ID NO: 35 - protein, 50 aa, Gallus gallus
MHEFQNLHSMYKARIQEFIRGHFYGHLDFSLENTLFFFIAGRYEFSNKGA

SEQ ID NO: 36 - protein, 50 aa, Bos taurus; identical to SEQ ID NO: 27

SEQ ID NO: 37 - protein, 50 aa, Aedes aegypti
IHEFQNLHAMAKEKIHEFTRGHFYGHFNFDLEKTLYMFIAGRYEFSNKGA

SEQ ID NO: 38 - protein, 50 aa, Drosophila melanogaster
IHEFQNLHAVAKEKINEFVRGHFYGHIDFDLDKTLYFFIAGRYEFGNKGA

SEQ ID NO: 39 - protein, 50 aa, Limulus polyphemus
LHEFQNLHALAKEKIHDFVRGHFYGHYDFDLDKTLYCFIAGRYEFSNKGA

SEQ ID NO: 40 - protein, 50 aa, Homo sapiens
VHEFQNLHAMYKARIQDFVRGHFYGHLDFDLEKTLFLFIAGRYEFFKTKG

SEQ ID NO: 41 - protein, 49 aa, Macaca mulatta
VHEFQNLHAMYKARIQDFVRGHFYGHLDFDLEKTLFLFIAGRYEFSNKG

SEQ ID NO: 42 - protein, 49 aa, Canis familiaris; identical to SEQ ID NO: 41

SEQ ID NO: 43 - protein, 49 aa, Danio rerio
MHEFQNLHSMNKSKIQEFVRGHFYGHLDFNLEKTLFFFIAGRYEFSNKG

SEQ ID NO: 44 - DNA, 16 bp, Equus caballus
ccctaccggaaggttc

SEQ ID NO: 45 - DNA, 21 bp, Equus caballus
ccctaccggaaggttccgggg

SEQ ID NO: 46 - DNA, 26 bp, Equus caballus
ggtcgcgcaccctaccggaaggttcc

SEQ ID NO: 47 - DNA, 11 bp, Equus caballus
accggaaggtt

SEQ ID NO: 48 - DNA, 15 bp, Equus caballus
ccctaccggaaggtt

SEQ ID NO: 49 - DNA, 34 bp, Equus caballus
ggtcgcgcaccctaccggaaggttccggggaatc

SEQ ID NO: 50 - DNA, 36 bp, Equus caballus
ggtcgcgcaccctaccggaaggttccggggaatcct

SEQ ID NO: 51 - DNA, 17 bp, Equus caballus
ccctaccggaaggttcc

SEQ ID NO: 52 - DNA, 11 bp, Equus caballus
accggaaggtt

(19) United States
(12) Patent Application Publication
(10) Pub. No.: US 2009/0258360 A1
(43) Pub. Date: Oct. 15, 2009

(54) METHOD OF DETECTING EQUINE POLYSACCHARIDE STORAGE MYOPATHY

(76) Inventors: Stephanie J. Valberg, Lino Lakes, MN (US); Molly E. McCue, Shoreview, MN (US); James R. Mickelson, Roseville, MN (US)

Correspondence Address: VIKSNINS HARRIS & PADYS PLLP, P.O. BOX 111098, ST. PAUL, MN, 55111-1098, US

(21) Appl. No.: 12/363,464

(22) Filed: Jan. 30, 2009

Related U.S. Application Data
(63) Continuation-in-part of application No. PCT/US2007/062134, filed on Feb. 14, 2007.

(51) Int. Cl. C12Q 1/68

(52) U.S. Cl. 435/6

(57) ABSTRACT

The present invention relates to diagnosing Polysaccharide Storage Myopathy (PSSM) disease in equines.

### PRIORITY OF INVENTION

The present patent application is a continuation-in-part application of Patent Cooperation Treaty Application No. PCT/US2007/062134 filed on Feb. 14, 2007. The present application claims the benefit of the above-listed application, which is hereby incorporated by reference herein in its entirety, including the drawings.

### BACKGROUND OF THE INVENTION

Polysaccharide Storage Myopathy (PSSM) is a debilitating muscle disease in many and diverse breeds of horses. Previous data indicates that approximately 10% of Quarter Horses and 36% of Belgian draft horses are affected. Clinical signs vary, but can range from muscle atrophy and progressive weakness in Draft horse breeds, to acute post-exercise muscle cramping and cell damage in Quarter Horses and other breeds. All forms of PSSM in horses are highly associated with deposits of an abnormal polysaccharide in skeletal muscle fibers that are demonstrated by histochemical staining of muscle biopsies. PSSM is also characterized by as much as four times the normal level of glycogen in skeletal muscle. Mutations in genes of glucose and glycogen metabolism are known to cause various types of glycogen storage diseases (glycogenoses) in humans and animal species, of which several histologically resemble PSSM. However, none of these genes appear to be responsible for equine PSSM.

The current diagnosis of PSSM in horses is based on clinical signs of muscle cramping or progressive atrophy (depending on the breed), often with elevated serum levels of muscle enzymes, combined with the histopathology finding of abnormal polysaccharide in thin sections cut from skeletal muscle biopsies.

Muscle biopsies are invasive, require skilled veterinary personnel to collect, are relatively expensive for the owner, and take a skilled muscle histopathologist to interpret. Further, although the muscle biopsy analysis has been a highly reliable diagnostic tool, it is not now 100% specific or sensitive, and can never hope to be.

Therefore, despite the foregoing, there is a need in the art for additional diagnostic tests for diagnosing PSSM in horses.

### SUMMARY OF THE INVENTION

The present invention features assays for determining the presence of a biomarker (e.g., a nucleic acid) associated with, or in linkage disequilibrium with, Polysaccharide Storage Myopathy (PSSM). In one embodiment, the method comprises determining whether a biomarker associated with the disease is present in a nucleic acid from the subject. In one embodiment the biomarker is a nucleic acid or gene in linkage disequilibrium with glycogen synthase enzyme 1 (GYS1). In certain embodiments, the PSSM associated allele biomarker is located in a region of equine chromosome-10 within 500 kb of GYS1 exon 6.

Appropriate biomarkers can be detected by any of a variety of means, including: 1) performing a hybridization reaction between the nucleic acid sample and a probe or probes that are capable of hybridizing to the biomarker; 2) sequencing at least a portion of the biomarker; or 3) determining the electrophoretic mobility of the biomarker or a component thereof. In one embodiment, the biomarker is a nucleic acid subject to an amplification step, prior to performance of the detection step. In certain embodiments, amplification steps are by polymerase chain reaction (PCR), ligase chain reaction (LCR), strand displacement amplification (SDA), cloning, and variations of the above (e.g., RT-PCR and allele specific amplification). In one embodiment, the sample is hybridized with a set of primers, which hybridize 5′ and 3′ to a sense or antisense sequence of a nucleic acid and is subject to PCR amplification.

In one embodiment, the detecting step is by allele specific hybridization followed by primer specific extension. In one embodiment, prior to or in conjunction with detection, the nucleic acid sample is subject to an amplification step. In one embodiment, the size analysis is preceded by a restriction enzyme digestion. In one embodiment, GYS1 or a portion thereof is amplified. In one embodiment, at least one oligonucleotide probe is immobilized on a solid surface.

In another aspect, the invention features kits for performing the above-described assays. The kit can include DNA sample collection means and a means for determining a biomarker that is indicative of PSSM in a horse. In one embodiment, the kit contains a first primer oligonucleotide that hybridizes 5′ or 3′ to a nucleic acid or gene in linkage disequilibrium with the mutant GYS1 allele that has a substitution of G to A at nucleotide 926 in exon 6. In one embodiment, the kit additionally comprises a second primer oligonucleotide that hybridizes either 3′ or 5′ respectively to the nucleic acid, so that the nucleic acid can be amplified. In one embodiment, first primer and the second primer hybridize to a region in the range of between about 50 and about 1000 base pairs. In one embodiment, the kit additionally comprises a detection means. In certain embodiments, the detection means is by a) allele specific hybridization; b) size analysis; c) sequencing; d) hybridization; e) 5′ nuclease digestion; f) single-stranded conformation polymorphism; g) primer specific extension; and/or h) oligonucleotide ligation assay. In certain embodiments, the kit additionally comprises an amplification means.

Information obtained using the assays and kits described herein is useful for determining whether a horse has or is susceptible to developing PSSM. In addition, the information allows customization of therapy to the horse's genetic profile.

The present invention provides a method for detecting the presence of a biomarker associated with equine Polysaccharide Storage Myopathy (PSSM). In one embodiment of the invention, the method involves obtaining a physiological sample from a horse, wherein the sample comprises nucleic acid, and determining the presence of the biomarker. As used herein, the phrase “physiological sample” is meant to refer to a biological sample obtained from a mammal that contains nucleic acid. For example, a physiological sample can be a sample collected from an individual horse, such as including, but not limited to, e.g., a cell sample, such as a blood cell, e.g., a lymphocyte, a peripheral blood cell; a sample collected from the spinal cord; a tissue sample such as cardiac tissue or muscle tissue, e.g., cardiac or skeletal muscle; an organ sample, e.g., liver or skin; a hair sample, e.g., a hair sample with roots; and/or a fluid sample, such as blood.

Examples of breeds of affected horse include, but are not limited to, Quarter Horses, Percheron Horses, Paint Horses, Draft Horses, Warmblood Horses, or other related or unrelated breeds. The phrase “related breed” is used herein to refer to breeds that are related to a breed, such as Quarter Horse, Draft Horse, or Warmblood Horse. Such breeds include, but are not limited to stock breeds such as the American Paint horse, the Appaloosa, and the Palomino. The term “Draft Horse” includes many breeds including but not limited to Clydesdale, Belgian, Percheron, and Shire horses. The term “Warmblood” is also a generic term that includes a number of different breeds. “Warmblood” simply distinguishes this type of horse from the “cold bloods” (draft horses) and the “hot bloods” (Thoroughbreds and Arabians). The method of the present invention also includes horses of crossed or mixed breeds.

The term “biomarker” is generally defined herein as a biological indicator, such as a particular molecular feature, that may affect or be related to diagnosing or predicting an individual's health. For example, in certain embodiments of the present invention, the biomarker comprises a mutant equine glycogen synthase enzyme 1 (GSY1) gene, such as a polymorphic allele of GYS1 has a substitution of G to A at nucleotide 926 in exon 6, or a nucleic acid positioned sufficiently close to the GYS1 gene to be in linkage disequilibrium with GYS1. The mutant GYS1 gene encodes a glycogen synthase enzyme having an R (arginine) to H (histidine) substitution at amino acid residue 309.

“Oligonucleotide probe” can refer to a nucleic acid segment, such as a primer, that is useful to amplify a sequence in the GYS1 gene that is complementary to, and hybridizes specifically to, a particular sequence in GYS1, or to a nucleic acid region that flanks GYS1 or to a nucleic acid positioned sufficiently close to the GYS1 gene to be in linkage disequilibrium with GYS1.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

In one embodiment of the present invention, the method also involves contacting the sample with at least one oligonucleotide probe to form a hybridized nucleic acid and amplifying the hybridized nucleic acid. “Amplifying” utilizes methods such as the polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. For example, in certain embodiments of the present invention, exon 6 of the equine glycogen synthase enzyme 1 gene (also referred to as GYS1), or a portion thereof, or a nucleic acid positioned sufficiently close to the GYS1 gene to be in linkage disequilibrium with GYS1 may be amplified by PCR. In another embodiment of the present invention, at least one oligonucleotide probe is immobilized on a solid surface.

The methods of the present invention can be used to detect the presence of a biomarker associated with equine Polysaccharide Storage Myopathy (PSSM) in a horse such as a foal, e.g., a neonatal foal or an aborted foal, one of a breeding pair of horses, e.g., the potential dam and/or sire, or any horse at any stage of life. The horse can be alive or dead.

Further provided by the present invention is a method for diagnosing Polysaccharide Storage Myopathy (PSSM) in a horse, the method involving obtaining a physiological sample from the horse, wherein the sample comprises nucleic acid; and detecting the presence of a biomarker in the sample, wherein the presence of the biomarker is indicative of the disease. One embodiment of the method further involves contacting the sample with at least one oligonucleotide probe to form a hybridized nucleic acid and amplifying the hybridized nucleic acid. For example, in one embodiment, exon 6 of equine glycogen synthase enzyme 1 or a portion thereof is amplified, for example, by polymerase chain reaction, strand displacement amplification, ligase chain reaction, amplification methods based on the use of Q-beta replicase and/or nucleic acid sequence-based amplification. In one embodiment of the method, the biomarker contains an equine glycogen synthase enzyme 1 gene having a G to A substitution at nucleotide 926 in exon 6 of the equine glycogen synthase enzyme 1 gene, or a gene encoding a glycogen synthase enzyme having an R to H substitution at amino acid residue 309. The method can be used to detect PSSM in a horse. In another embodiment a nucleic acid in linkage disequilibrium with GYS1 is amplified.

Further provided by the present invention is a kit comprising a diagnostic test for detecting the presence of equine PSSM in a horse comprising packaging material, containing, separately packaged, at least one oligonucleotide probe capable of forming a hybridized nucleic acid with GYS1 or a nucleic acid positioned sufficiently close to the GYS1 gene to be in linkage disequilibrium with GYS1, and instructions means directing the use of the probe in accord with the methods of the invention.

Other features and advantages of the invention will be apparent from the following detailed description and claims.

### BRIEF DESCRIPTION OF DRAWINGS

FIG. 1. Normal Equine GYS1 Coding DNA Sequence (SEQ ID NO:1). Exon 6 is indicated in bold. The site of a G to A mutation site at nucleotide position 926 is underlined. This region of sequence is expanded below in FIG. 2.

FIG. 2. GYS1 Exon 6 and Flanking DNA Sequence from Normal (SEQ ID NO:2) and PSSM Horses (SEQ ID NO:3). Exon 6 in these equine GYS1 DNA sequences contains positions 33-150. At position 135 a G in the normal horse sequence is replaced by an A in the PSSM horse sequence. This changes the underlined three base codon from one coding for an arginine (CGT) to one coding for a histidine (CAT).

FIG. 3. Glycogen Synthase Amino Sequences Encoded by Exon 6 of the GYS1 Genes. Species included in the analysis are Human, Control Horse, Chimpanzee, Canine, Bovine, Mouse, Rat, Pig, and Zebrafish. All species have identical amino acid sequences in this region of the skeletal muscle glycogen synthase protein (SEQ ID NO:4), which represents the 39 amino acids encoded by nucleotide positions 33-150 in the DNA sequences of FIG. 2. However, PSSM horses have a histidine (H) at amino acid position 34 in this exon (underlined) (SEQ ID NO:5), while all other species have an arginine (R). This codon represents number 309 in the complete coding sequence.

FIG. 4. Horse GYS1 Intron 5, Exon 6, and Intron 6 genomic DNA sequence from which PCR primers to amplify the PSSM GYS1 mutation would be most appropriately derived (SEQ ID NO:6). Exon 6 is indicated in bold.

FIG. 5. The entire GYS1 coding nucleotide sequence in FIG. 1 was translated to give this amino acid sequence (SEQ ID NO:9). The site of the R to H mutation at codon 309 is underlined.

FIGS. 6a-6f: Extended PSSM pedigrees. Extended pedigrees from the three PSSM families used for the related case cohort in the whole genome association. Square symbols denote males, circles denote females, filled symbols denote PSSM affected based on histopathology, X through the symbol indicates not tested, N indicates phenotyped normal based on histopathology, arrows at top right of symbol indicate horses that demonstrated signs consistent with PSSM based on owner/breeder interview (not phenotyped by biopsy). FIGS. 6a, 6b, 6c, and 6d represent a single large extended family. Individuals circled in 2a represent founders of the subsequent pedigrees, 6b, 6c and 6d. FIGS. 6e and 6f represent two additional extended families from which individuals were selected for inclusion in the case cohort. FIG. 6a: 8 phenotyped individuals, 7 with PSSM originating from EC. Individuals circled represent founders of the subsequent pedigrees, 6b, 6c and 6d. FIG. 6b: 37 phenotyped individuals, 22 with PSSM originating from GF a grandson of EC. FIG. 6c: 8 phenotyped individuals, 8 with PSSM originating from GE a grandson of EC. FIG. 6d: 15 phenotyped individuals, 10 with PSSM originating from LBE or EBR, full siblings who are grandsons of EC. FIG. 6e: 17 phenotyped individuals, 15 with PSSM originating from SDB. FIG. 6f: 18 phenotyped individuals, 12 with PSSM originating from PI.

FIG. 7: Range of p values from chi-square test of association for initial 105 microsatellites. Histogram depicting the range of p values from all 105 MS markers. Values included in each bin range from the label value to < the value of the following bin. For example bin 0.0001 includes all MS with a p value of 0.0001 to <0.001.

FIG. 8: ECA10p p values. Map of ECA10p and p values association of MS marker alleles with PSSM in the initial and follow-up case and control cohorts. From left to right: idiogram of ECA10p with FISH-anchored loci (21); Radiation hybrid map of ECA10p with comparatively mapped loci to HSA19 (21); p values for chi-squared comparison of MS allele frequencies in the initial population and the follow-up population. The GYS1 gene at the 54.1 Mb position on HSA19 is indicated.

FIG. 9: Glycogen Synthase amino acid alignment. Multiple amino acid alignment of glycogen synthase 1 (skeletal muscle isoform) and glycogen synthase 2 (liver isoform). Residue 309 at the site of the GYS1 PSSM mutation, and the surrounding amino acids, are highly conserved across species and across both GS isoforms.

FIG. 10: Glycogen Synthase Activity. Mean GS activity in the presence and absence of its allosteric activator, glucose 6-phosphate (G6P). * indicates significant difference between PSSM and controls.

FIG. 11: Conserved SNP haplotypes among breeds containing the GYS1 A (PSSM) allele. Minimally conserved haplotype in all breeds is highlighted and the length in Kb in different breeds indicated on the right. The Kb on the horizontal position is the relative position of each SNP within the 2 Mb contig containing the GYS1 gene. The GYS1 A allele producing the Arg309His GS mutation is indicated at the 819 Kb position as the PSSMSNPEXON6 SNP.

FIG. 12: Segregation of GYS1 genotypes with the PSSM phenotype. The pedigree is from a multi-year breeding trial at the University of Minnesota. Filled symbols represent individuals with PSSM based on the presence of abnormal polysaccharide in skeletal muscle fibers and open symbols indicate a normal biopsy result. The Arg309His genotype of each horse is provided beneath its symbol.

FIG. 13: ECA10p p values R/R PSSM. P values for chi square test of association between MS markers and PSSM phenotype in R/R.

FIG. 14: Sequenom® SNPs. Position and relative distance from GYS1 of ECA10p SNPs used in Sequenom® assay.

FIG. 15: Allele age estimation. Estimations of allele frequency, population growth rate, and proportion of disease chromosomes sampled, and calculation of weighted averages used in allele age approximation.
^{a} estimates obtained from the American Quarter Horse Association website.
^{b} estimates obtained from the American Paint Horse Association website.
^{c} estimates obtained from Belgian Draft Horse Cooperation of America.
^{d} estimates obtained from Percheron Horse Association of America.
^{e} estimates from genotype data from 100 random hair root samples of each breed.
^{f} number of chromosomes from each breed with A allele included in the data set for analysis.

FIG. 16: Genes in linkage disequilibrium with GYS1. As determined from the world-wide-web at ncbi.nlm.nih.gov/sites/entrez?db=gene&cmd=retrieve&dopt=full_report&list_uids=100055178.

### DETAILED DESCRIPTION OF THE INVENTION

Horses affected with Polysaccharide Storage Myopathy (PSSM) are typically heterozygous for the mutant allele of the GYS1 gene.

Genotype Screening

Traditional methods for the screening of heritable diseases have depended on either the identification of abnormal gene products (e.g., sickle cell anemia) or an abnormal phenotype (e.g., mental retardation). With the development of simple and inexpensive genetic screening methodology, it is now possible to identify polymorphisms that indicate a propensity to develop disease, even when the disease is of polygenic origin.

Genetic screening (also called genotyping or molecular screening), can be broadly defined as testing to determine if a patient has mutations (or alleles or polymorphisms) that either cause a disease state or are “linked” to the mutation causing a disease state. Linkage refers to the phenomenon that DNA sequences which are close together in the genome have a tendency to be inherited together. The closer they are to the GYS1 gene, the stronger the likelihood of co-inheritance. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a haplotype which spans or is linked to a disease-causing mutational change, serves as a predictive measure of an individual's likelihood of having inherited that disease-causing mutation. Such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases.

The statistical correlation between a disorder and a polymorphism does not necessarily indicate that the polymorphism directly causes the disorder. Rather, the correlated polymorphism may be a benign allelic variant which is linked to (i.e., in linkage disequilibrium with) the true a disorder-causing mutation which has occurred in the recent evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. A broad-spanning haplotype (describing the typical pattern of co-inheritance of alleles of a set of linked polymorphic markers) can be targeted for diagnostic purposes once an association has been drawn between a particular disease or condition and a corresponding haplotype. Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

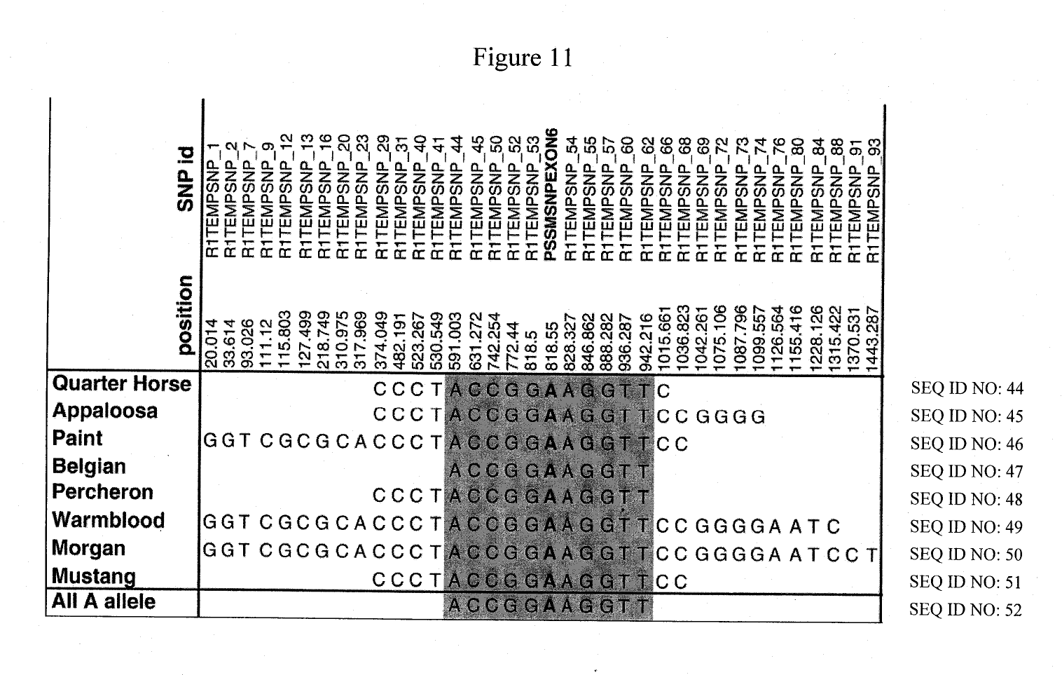

This principle is illustrated in FIG. 11, where a number of SNP DNA markers over a several Mb region of equine chromosome 10, in the region of the GYS1 gene are indicated. The specific alleles of each SNP marker that form a haplotype in eight different breeds are identified. It is seen that all PSSM horses share a minimal haplotype of approximately 357 kb. This means that any SNP marker or combination of SNP markers within the 357 kb DNA fragment will be in linkage disequilibrium to the GYS1 mutation and can be diagnostic of the PSSM GYS1 mutation.

Definitions

An “allele” is a variant form of a particular gene. For example, the present invention relates, inter alia, to the discovery that some alleles of the GYS1 gene cause PSSM in horses. A “GYS1 allele” refers to a normal allele of the GYS1 locus as well as an allele carrying a variation(s) that predispose a horse to develop PSSM. The coexistence of multiple alleles at a locus is known as “genetic polymorphism.” Any site at which multiple alleles exist as stable components of the population is by definition “polymorphic.” An allele is defined as polymorphic if it is present at a frequency of at least 1% in the population. A “single nucleotide polymorphism (SNP)” is a DNA sequence variation that involves a change in a single nucleotide.

“Biological activity” or “bioactivity” or “activity” or “biological function,” which are used interchangeably, for the purposes herein means an effector or antigenic function that is directly or indirectly performed by an GYS1 polypeptide (whether in its native or denatured conformation), or by any subsequence thereof. Biological activities include binding to a target peptide, e.g., an receptor. A GYS1 bioactivity can be modulated by directly affecting a GYS1 polypeptide. Alternatively, an GYS1 bioactivity can be modulated by modulating the level of a GYS1 polypeptide, such as by modulating expression of a GYS1 gene.

As used herein the term “bioactive fragment of a GYS1 polypeptide” refers to a fragment of a full-length GYS1 polypeptide, wherein the fragment specifically mimics or antagonizes the activity of a wild-type GYS1 polypeptide.

The term “an aberrant activity,” as applied to an activity of a polypeptide such as GYS1, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant GYS1 activity due to over-expression or under-expression of a GYS1 locus gene encoding a GYS1 locus polypeptide.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, in certain embodiments, the control sample comprises the material to be tested. However, where the sample to be tested is genomic DNA, the control sample is preferably a highly purified sample of genomic DNA.

“Genotyping” refers to the analysis of an individual's genomic DNA (or a nucleic acid corresponding thereto) to identify a particular disease causing or contributing mutation or polymorphism, directly or based on detection of a mutation or polymorphism (a marker) that is in linkage disequilibrium with the disease causing or contributing gene.

The term “haplotype” as used herein is intended to refer to a set of alleles that are inherited together as a group (are in linkage disequilibrium) at statistically significant levels (p_{corr}<0.05). As used herein, the phrase “an GYS1 haplotype” refers to a haplotype in the GYS1 loci.

“Increased risk” refers to a statistically higher frequency of occurrence of the disease or condition in an individual carrying a particular polymorphic allele in comparison to the frequency of occurrence of the disease or condition in a member of a population that does not carry the particular polymorphic allele.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium.” The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern.

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene.” A specific genetic sequence at a polymorphic region of a gene is an allele. A polymorphic region can be a single nucleotide, the identity of which differs in different alleles. A polymorphic region can also be several nucleotides long.

The term “propensity to disease,” also “predisposition” or “susceptibility” to disease or any similar phrase, means that certain alleles are hereby discovered to be associated with or predictive of a subject's incidence of developing a particular disease (e.g., exercise induced collapse). The alleles are thus over-represented in frequency in individuals with disease as compared to healthy individuals. Thus, these alleles can be used to predict disease even in pre-symptomatic or pre-diseased individuals.

As used herein, the term “specifically hybridizes” or “specifically detects” refers to the ability of a nucleic acid molecule to hybridize to at least approximately six consecutive nucleotides of a sample nucleic acid.

The terms “protein,” “peptide” and “polypeptide” are used interchangeably herein.

The invention encompasses isolated or substantially purified nucleic acid compositions. In the context of the present invention, an “isolated” or “purified” DNA molecule is a DNA molecule that, by human intervention, exists apart from its native environment and is therefore not a product of nature. An isolated DNA molecule may exist in a purified form or may exist in a non-native environment. For example, an “isolated” or “purified” nucleic acid molecule, or portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences and proteins or partial-length proteins encoded thereby are also encompassed by the present invention.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Synthetic” polynucleotides are those prepared by chemical synthesis.

“Recombinant DNA molecule” is a combination of DNA sequences that are joined together using recombinant DNA technology and procedures used to join together DNA sequences as described, for example, in Sambrook and Russell (2001).

The term “gene” is used broadly to refer to any segment of nucleic acid associated with a biological function. Genes include coding sequences and/or the regulatory sequences required for their expression. For example, gene refers to a nucleic acid fragment that expresses MRNA, functional RNA, or a specific protein, such as glycogen synthase enzyme 1, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons. “Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

A “mutant” glycogen synthase enzyme 1 (GYS1) refers to the protein or fragment thereof that is encoded by a GYS1 gene having a mutation, e.g., such as might occur at the GYS1 locus. A mutation in one GYS1 allele may lead to enhanced or increased enzymatic activity in a horse heterozygous for the allele. Increased enzymatic activity can be determined by methods known to the art. Mutations in GYS1 may be disease-causing in a horse heterozygous for the mutant GYS1 allele, e.g., a horse heterozygous for a mutation leading to a mutant gene product such as a substitution mutation in exon 6 of GYS1, such as that designated herein as G926A.

“Somatic mutations” are those that occur only in certain tissues, e.g., in liver tissue, and are not inherited in the germline. “Germline” mutations can be found in any of a body's tissues and are inherited. The present GYSE1 mutation is a germline mutation.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, or at least 80%, 90%, or even at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%; at least 80%, 81%, 82%, 83%, 84%, 85%, 86%, 87%, 88%, or 89%; or at least 90%, 91%, 92%, 93%, or 94%; or even at least 95%, 96%, 97%, 98% or 99% sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl:

Tₘ81.5° C.+16.6 (log M)+0.41 (%GC)−0.61 (% form)−500/L

where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2X SSC wash at 65° C. for 15 minutes. Often, a high stringency wash is preceded by a low stringency wash to remove background probe signal. An example medium stringency wash for a duplex of, e.g., more than 100 nucleotides, is 1X SSC at 45° C. for 15 minutes. An example low stringency wash for a duplex of, e.g., more than 100 nucleotides, is 4-6X SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1X SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1X to 2X SSC (20X SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5X to 1X SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. Guidance as to appropriate amino acid substitutions that do not affect biological activity of the protein of interest are well known in the art. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally-occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations.”

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.”

A “host cell” is a cell which has been transformed, or is capable of transformation, by an exogenous nucleic acid molecule. Thus, “transformed,” “transgenic,” and “recombinant” refer to a host cell or organism into which a heterologous nucleic acid molecule has been introduced. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically includes sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will have the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The transcriptional cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source.

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified through, for example, the use of single-stranded mutagenesis. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Genome” refers to the complete genetic material of an organism.

“Coding sequence” refers to a DNA or RNA sequence that codes for a specific amino acid sequence and excludes the non-coding sequences. For example, a DNA “coding sequence” or a “sequence encoding” a particular polypeptide, is a DNA sequence which is transcribed and translated into a polypeptide in vitro or in vivo when placed under the control of appropriate regulatory elements. The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3 ′ to the coding sequence. It may constitute an “uninterrupted coding sequence,” i.e., lacking an intron, such as in a cDNA or it may include one or more introns bounded by appropriate splice junctions. An “intron” is a sequence of RNA that is contained in the primary transcript but that is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (MRNA translation).

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

The term “regulatory sequence” is art-recognized and intended to include promoters, enhancers and other expression control elements (e.g., polyadenylation signals). Such regulatory sequences are known to those skilled in the art. It should be understood that the design of the expression vector may depend on such factors as the choice of the host cell to be transfected and/or the amount of fusion protein to be expressed.

The term DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

A control element, such as a promoter, “directs the transcription” of a coding sequence in a cell when RNA polymerase binds the promoter and transcribes the coding sequence into mRNA, which is then translated into the polypeptide encoded by the coding sequence.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones having a population of daughter cells containing the exogenous DNA.

“Operably-linked” refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” or “translation stop codon” or “stop codon” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. The change of at least one nucleotide in a nucleic acid sequence can result in an interruption of the coding sequence of the gene, e.g., a premature stop codon. Such sequence changes can cause a mutation in the polypeptide encoded by a GYS1 gene. For example, if the mutation is a nonsense mutation, the mutation results in the generation of a premature stop codon, causing the generation of a truncated GYS polypeptide.

Prognostic Assays and Kits

The invention is based, at least in part, on the findings, which are described in detail in the following examples, that the GYS 1 with a substitution of G to A at nucleotide 926 in exon 6 (G926A) is significantly associated with the development of PSSM. The present invention, therefore, provides methods and kits for determining whether a subject has or is or is a carrier for PSSM.

In addition to the allelic patterns described above, as described herein, one of skill in the art can readily identify other alleles (including polymorphisms and mutations) that are in linkage disequilibrium with an allele associated with PSSM. For example, a nucleic acid sample from a first group of subjects without a particular disorder can be collected, as well as DNA from a second group of subjects with the disorder. The nucleic acid sample can then be compared to identify those alleles that are over-represented in the second group as compared with the first group, wherein such alleles are presumably associated with a disorder. Alternatively, alleles that are in linkage disequilibrium with an allele that is associated with the disorder can be identified, for example, by genotyping a large population and performing statistical analysis to determine which alleles appear more commonly together than expected. The group may be chosen to be comprised of genetically related individuals. Genetically related individuals include individuals from the same breed, or even the same family. As the degree of genetic relatedness between a control group and a test group increases, so does the predictive value of polymorphic alleles that are ever more distantly linked to a disease-causing allele. This is due to the fact that less evolutionary time has passed to allow polymorphisms which are linked along a chromosome in a founder population to redistribute through genetic cross-over events. Thus breed-specific, and even family-specific diagnostic genotyping assays can be developed to allow for the detection of disease alleles which arose at ever more recent times in equine evolution.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common breed or family, the reliability of ever more distantly spaced polymorphic loci can be relied upon as an indicator of inheritance of a linked disease-causing mutation.

In another embodiment, the method of the invention may be employed by detecting the presence of a GYS1 associated polymorphism that is in linkage disequilibrium with one or more predictive alleles. Alleles of the GYS1 haplotype are known to be in linkage disequilibrium are the genes and intergenic regions between the 18,700,000 and 19,100,000 basepair position of equine chromosome 10. For example, see the genes shown in FIG. 16.

Appropriate probes may be designed to hybridize to a specific gene of the GYS1 locus. Alternatively, these probes may incorporate other regions of the relevant genomic locus, including intergenic sequences. Yet other polymorphisms available for use with the immediate invention are obtainable from various public sources. From such sources SNPs as well as other equine polymorphisms may be found.

Accordingly, the nucleotide segments of the invention may be used for their ability to selectively form duplex molecules with complementary stretches of equine chromosomes or cDNAs from that region or to provide primers for amplification of DNA or cDNA from this region. The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as a molecular probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCl at temperatures of about 50° C. to about 70° C. Such selective conditions may tolerate little, if any, mismatch between the probe and the template or target strand.

Other alleles or other indicia of a disorder can be detected or monitored in a subject in conjunction with detection of the alleles described above.

Many methods are available for detecting specific alleles at equine polymorphic loci. Certain methods for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

Nucleic Acids of the Invention

Sources of nucleotide sequences from which the present nucleic acid molecules can be obtained include any prokaryotic or eukaryotic source. For example, they can be obtained from a mammalian, such as an equine, cellular source. Alternatively, nucleic acid molecules of the present invention can be obtained from a library, such as the CHORI-241 Equine BAC library or the BAC library developed at INRA, Centre de Recherches de Jouy, Laboratoire de Génétique biochimique et de Cytogénétique, Département de Génétique animale, 78350 Jouy-en-Josas Cedex, France.

As discussed above, the terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule that is complementary or hybridizes to a sequence in a gene of interest, i.e., a nucleic acid sequence encoding an equine glycogen synthase enzyme, and remains stably bound under stringent conditions (as defined by methods well known in the art). Thus, the RNA or DNA is “isolated” in that it is free from at least one contaminating nucleic acid with which it is normally associated in the natural source of the RNA or DNA and in one embodiment of the invention is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

Nucleic Acid Amplification Methods

According to the methods of the present invention, the amplification of DNA present in a physiological sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the Qβ replicase system, nucleic acid sequence-based amplification (or “NASBA”), the repair chain reaction (or “RCR”), and boomerang DNA amplification (or “BDA”).

The bases incorporated into the amplification product may be natural or modified bases (modified before or after amplification), and the bases may be selected to optimize subsequent electrochemical detection steps.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques. Where the nucleic acid to be amplified is RNA, amplification may be carried out by initial conversion to DNA by reverse transcriptase in accordance with known techniques.

Strand displacement amplification (SDA) may be carried out in accordance with known techniques. For example, SDA may be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide probe of the present invention) that hybridizes to the target sequence to be amplified and/or detected. The flanking sequence, which serves to facilitate binding of the restriction enzyme to the recognition site and provides a DNA polymerase priming site after the restriction site has been nicked, is about 15 to 20 nucleotides in length in one embodiment. The restriction site is functional in the SDA reaction. The oligonucleotide probe portion is about 13 to 15 nucleotides in length in one embodiment of the invention.

Ligase chain reaction (LCR) is also carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

In one embodiment of the invention, each exon of the GYS1 gene is amplified by PCR using primers based on the known sequence. The amplified exons are then sequenced using automated sequencers. In this manner, the exons of the GYS1 gene from horses suspected of having PSSM in their pedigree are sequenced until a mutation is found. Examples of such mutations include those in exon 6 of the GYS1 DNA. For example, one mutation is the G to A substitution at nucleotide base 926 in exon 6. Using this technique, additional mutations causing equine PSSM can be identified.

According to the diagnostic method of the present invention, alteration within the wild-type GYS1 locus is detected. “Alteration of a wild-type gene” encompasses all forms of mutations including deletions, insertions and point mutations in the coding and noncoding regions. Deletions may be of the entire gene or of only a portion of the gene. Point mutations may result in stop codons, frameshift mutations or amino acid substitutions. Point mutational events may occur in regulatory regions, such as in the promoter of the gene, leading to loss or diminution of expression of the MRNA. Point mutations may also abolish proper RNA processing, leading to loss of expression of the GYS1 gene product, or to a decrease in mRNA stability or translation efficiency. PSSM is a disease caused by a point mutation at nucleic acid 926. Horses predisposed to or have PSSM only need to have one mutated allele.

Diagnostic techniques that are useful in the methods of the invention include, but are not limited to direct DNA sequencing, PFGE analysis, allele-specific oligonucleotide (ASO), dot blot analysis and denaturing gradient gel electrophoresis, and are well known to the artisan.

There are several methods that can be used to detect DNA sequence variation. Direct DNA sequencing, either manual sequencing or automated fluorescent sequencing can detect sequence variation. Another approach is the single-stranded conformation polymorphism assay (SSCA). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection on a research basis. The fragments that have shifted mobility on SSCA gels are then sequenced to determine the exact nature of the DNA sequence variation. Other approaches based on the detection of mismatches between the two complementary DNA strands include clamped denaturing gel electrophoresis (CDGE), heteroduplex analysis (HA) and chemical mismatch cleavage (CMC). Once a mutation is known, an allele specific detection approach such as allele specific oligonucleotide (ASO) hybridization can be utilized to rapidly screen large numbers of other samples for that same mutation. Such a technique can utilize probes which are labeled with gold nanoparticles to yield a visual color result.

Detection of point mutations may be accomplished by molecular cloning of the GYS1 allele(s) and sequencing the allele(s) using techniques well known in the art. Alternatively, the gene sequences can be amplified directly from a genomic DNA preparation from equine tissue, using known techniques. The DNA sequence of the amplified sequences can then be determined.

There are six well known methods for a more complete, yet still indirect, test for confirming the presence of a mutant allele: 1) single stranded conformation analysis (SSCA); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. For allele-specific PCR, primers are used which hybridize at their 3′ ends to a particular GYS1 mutation. If the particular mutation is not present, an amplification product is not observed. Amplification Refractory Mutation System (ARMS) can also be used. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Other techniques for detecting insertions and deletions as known in the art can be used.

In the first three methods (SSCA, DGGE and RNase protection assay), a new electrophoretic band appears. SSCA detects a band that migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. In the practice of the present invention, the method involves the use of a labeled riboprobe that is complementary to the horse wild-type GYS1 gene coding sequence. The riboprobe and either mRNA or DNA isolated from the tumor tissue are annealed (hybridized) together and subsequently digested with the enzyme RNase A that is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the GYS1 mRNA or gene but can be a segment of either. If the riboprobe comprises only a segment of the GYS1 mRNA or gene, it will be desirable to use a number of these probes to screen the whole mRNA sequence for mismatches.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA that might contain a mutation can be amplified using PCR before hybridization.

Nucleic acid analysis via microchip technology is also applicable to the present invention.

DNA sequences of the GYS1 gene that have been amplified by use of PCR may also be screened using allele-specific probes. These probes are nucleic acid oligomers, each of which contains a region of the GYS1 gene sequence harboring a known mutation. For example, one oligomer may be about 30 nucleotides in length, corresponding to a portion of the GYS1 gene sequence. By use of a battery of such allele-specific probes, PCR amplification products can be screened to identify the presence of a previously identified mutation in the GYS1 gene. Hybridization of allele-specific probes with amplified GYS1 sequences can be performed, for example, on a nylon filter. Hybridization to a particular probe under stringent hybridization conditions indicates the presence of the same mutation in the tissue as in the allele-specific probe.

Alteration of GYS1 mRNA expression can be detected by any technique known in the art. These include Northern blot analysis, PCR amplification and RNase protection. Diminished mRNA expression indicates an alteration of the wild-type GYS1 gene.

Alteration of wild-type GYS1 genes can also be detected by screening for alteration of wild-type GYS1 protein, or a portion of the GYS1 protein. For example, monoclonal antibodies immunoreactive with GYS1 (or to a specific portion of the GYS1 protein) can be used to screen a tissue. Lack of cognate antigen would indicate a mutation. Antibodies specific for products of mutant alleles could also be used to detect mutant GYS1 gene product. Such immunological assays can be done in any convenient formats known in the art. These include Western blots, immunohistochemical assays and ELISA assays. Any means for detecting an altered GYS1 protein can be used to detect alteration of wild-type GYS1 genes. Functional assays, such as protein binding determinations, can be used. In addition, assays can be used that detect GYS1 biochemical function. Finding a mutant GYS1 gene product indicates alteration of a wild-type GYS1 gene.

Mutant GYS1 genes or gene products can be detected in a variety of physiological samples collected from a horse. Examples of appropriate samples include a cell sample, such as a blood cell, e.g., a lymphocyte, a peripheral blood cell; a sample collected from the spinal cord; a tissue sample such as cardiac tissue or muscle tissue, e.g., cardiac or skeletal muscle; an organ sample, e.g., liver or skin; a hair sample, especially a hair sample with roots; a fluid sample, such as blood.

The methods of diagnosis of the present invention are applicable to any equine disease in which GYS1 has a role. The diagnostic method of the present invention is useful, for example, for veterinarians, Breed Associations, or individual breeders, so they can decide upon an appropriate course of treatment, and/or to determine if an animal is a suitable candidate as a broodmare or sire.

Oligonucleotide Probes

As noted above, the method of the present invention is useful for detecting the presence of a polymorphism in equine DNA, in particular, the presence of a G to A nucleotide substitution at position 926 in exon 6 of the coding sequence of equine GYS1 (SEQ ID NO:1). This substitution results in the replacement of an arginine (R) amino acid at codon 309 by a histidine (H) in the glycogen synthase protein (SEQ ID NO:9).

Primer pairs are useful for determination of the nucleotide sequence of a particular GYS1 allele using PCR. The pairs of single-stranded DNA primers can be annealed to sequences within or surrounding the GYS1 gene in order to prime amplifying DNA synthesis of the GYS1 gene itself. A complete set of these primers allows synthesis of all of the nucleotides of the GYS1 coding sequences, i.e., the exons. The set of primers preferably allows synthesis of both intron and exon sequences. Allele-specific primers can also be used. Such primers anneal only to particular GYS1 mutant alleles, and thus will only amplify a product in the presence of the mutant allele as a template.

The first step of the process involves contacting a physiological sample obtained from a horse, which sample contains nucleic acid, with an oligonucleotide probe to form a hybridized DNA. The oligonucleotide probes that are useful in the methods of the present invention can be any probe comprised of between about 4 or 6 bases up to about 80 or 100 bases or more. In one embodiment of the present invention, the probes are between about 10 and about 20 bases.

The primers themselves can be synthesized using techniques that are well known in the art. Generally, the primers can be made using oligonucleotide synthesizing machines that are commercially available. Given the sequence of the GYS1 coding sequence as set forth in SEQ ID NO:1, design of particular primers is well within the skill of the art.

Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-(3-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science 254, 1497-1500 (1991).

The only requirement is that the oligonucleotide probe should possess a sequence at least a portion of which is capable of binding to a known portion of the sequence of the DNA sample.

It may be desirable in some applications to contact the DNA sample with a number of oligonucleotide probes having different base sequences (e.g., where there are two or more target nucleic acids in the sample, or where a single target nucleic acid is hybridized to two or more probes in a “sandwich” assay).

The nucleic acid probes provided by the present invention are useful for a number of purposes. The probes can be used to detect PCR amplification products. They may also be used to detect mismatches with the GYS1 gene or mRNA using other techniques.

Hybridization Methodology

The DNA (or nucleic acid) sample may be contacted with the oligonucleotide probe in any suitable manner known to those skilled in the art. For example, the DNA sample may be solubilized in solution, and contacted with the oligonucleotide probe by solubilizing the oligonucleotide probe in solution with the DNA sample under conditions that permit hybridization. Suitable conditions are well known to those skilled in the art. Alternatively, the DNA sample may be solubilized in solution with the oligonucleotide probe immobilized on a solid support, whereby the DNA sample may be contacted with the oligonucleotide probe by immersing the solid support having the oligonucleotide probe immobilized thereon in the solution containing the DNA sample.

### EXAMPLE 1

### Method of Detecting a DNA Mutation Associated with Equine Polysaccharide Storage Myopathy

The present invention relates to mutations in the GYS1 gene and their use in the diagnosis of PSSM, the diagnosis of predisposition to PSSM, and to the detection of a mutant GYS1 allele in a horse.

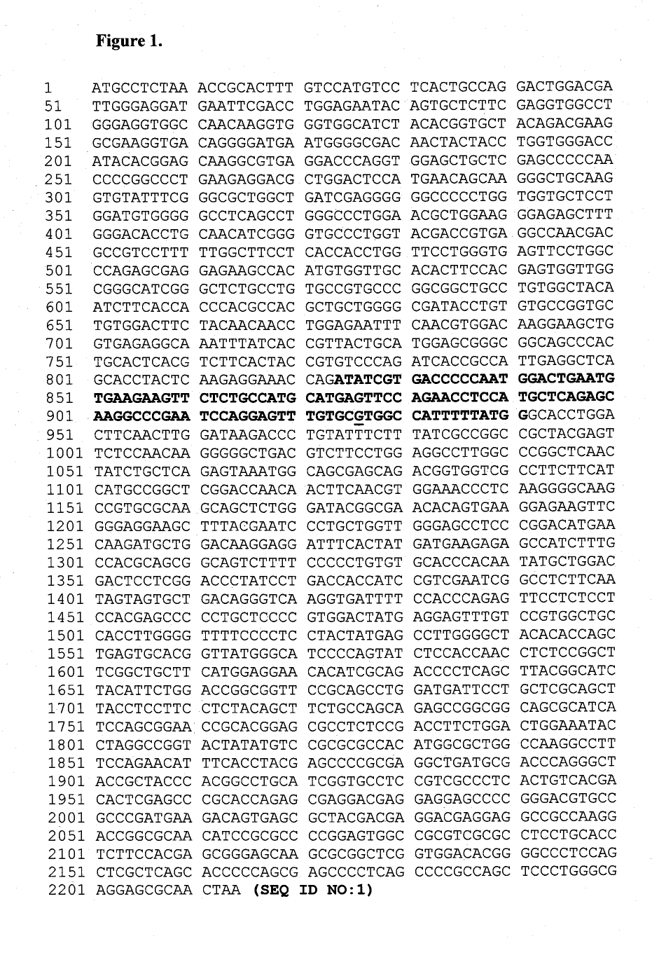

The present inventors discovered a mutation in the equine GYS1 gene (encoding the skeletal muscle glycogen synthase enzyme) that is present in many populations of PSSM affected horses studied to date. This was possible by first deriving the protein-encoding DNA sequence of the equine GYS1 gene from mRNA isolated from skeletal muscle of both an affected and a control horse. In both horses the sequence length from the start codon (ATG) to the stop codon (TAA) was 2,214 bases (FIG. 1) and would code for a protein of 737 amino acids. The only difference between the PSSM and control horse sequences was a G to A base substitution in exon 6 at nucleotide position 926.

The DNA sequence difference at position 926 of the GYS1 coding sequence present in skeletal muscle mRNA was subsequently confirmed in the genomic DNA of several horses. An expanded view of exon 6 with its flanking intron sequence from genomic DNA is shown in FIG. 2. FIG. 2 also shows that the change from a G to A in the DNA sequence causes the replacement of an arginine (R) amino acid at codon 309 by a histidine (H) in the glycogen synthase protein. Thus, this mutation may be referred to as the G926 to A926 DNA mutation or the R309 to H309 amino acid mutation. The normal alleles of this gene may be referred to as G926, R or R309, and the mutant alleles as A926, H or H309.

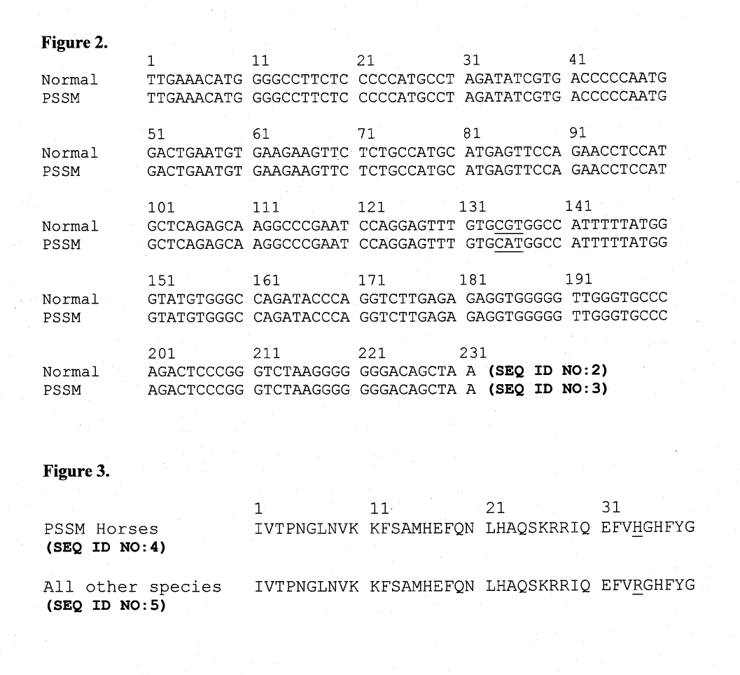

To date, no other mutations in the GYS1 gene have been shown to cause a glycogen storage disease in humans or animal species. The related GYS2 gene, encoding the liver form of glycogen synthase that is expressed in non-muscle tissues, has several known mutations that lead to a deficiency in this enzyme and fasting hypoglycemia. However, unlike the GYS2 mutations that greatly reduce the activity of the glycogen synthase enzyme and are inherited in a recessive manner, the PSSM horse muscle GYS1 mutation does not reduce the glycogen synthase activity. Rather, it appears to result in an increased glycogen synthase activity and be inherited in a dominant fashion (see Table 1 below). This region of the muscle glycogen synthase amino acid sequence contained in exon 6 is highly conserved throughout the animal kingdom, lending support to its mutation in PSSM horses being a causative mutation (FIG. 3).

The inventors have found the GYS1 R to H mutation in PSSM-affected Quarter Horses, Draft horses, and Warmbloods (Table 1), and it is likely to extend to even more breeds of horses. Approximately 80% of the Quarter Horses and Belgian Draft Horses diagnosed with PSSM by the muscle biopsy method thus far are either homozygous (have two copies of the H allele; H/H) or are heterozygous (an H and an R allele; R/H). PSSM horses with the GYS1 H allele can be of either sex, and this is consistent with, but does not prove an autosomal dominant inheritance. Only 4% of Quarter Horses and 14% of the Belgian Draft Horses with negative biopsy results were heterozygous. The inventors believe this in large part reflects the less than 100% accuracy of the current diagnostic method, but could also reflect an incomplete penetrance; i.e., carriers of the H allele may not always develop disease symptoms due to other genetic and environmental factors.

TABLE 1
GYS1 Genotype Frequencies in PSSM and
Control Horses of Different Breeds

| | | | | | PSSM | Control |
|---|---|---|---|---|---|---|
| | PSSM | Control | PSSM | Control | Warm- | Warm- |
| Genotype | QH | QH | Belgian | Belgian | blood | blood |
| R/R | 18 | 85 | 4 | 29 | 1 | 4 |
| R/H | 67 | 4 | 28 | 5 | 3 | 0 |
| H/H | 4 | 0 | 4 | 0 | 0 | 0 |

That approximately 20% of the Quarter Horses and 11% of Belgians with abnormal polysaccharide in muscle biopsies and clinical signs of PSSM do not carry the GYS1 H allele is suggestive that there may be other causes of PSSM. In other words, the GYS1 mutation appears to explain most, but not all cases of equine PSSM, and there is likely to be another gene responsible for a non-GYS1 form of PSSM that will be need to be the subject of additional investigation.

The inventors have determined the GYS1 genotype frequency in random populations of horses obtained from samples submitted for the purposes of breed registration requirements. Hair root samples were taken from every 10^{th} submission to ensure even distribution across the US. Table 2 indicates that the GYS1 mutation is very prevalent in four major breeds examined to date, but not yet in Thoroughbreds. The GYS1 genotype distribution in Quarter Horses and Paint Horses is similar at 6-7% heterozygous with few homozygotes for the H allele. However, approximately 42% of Percherons are heterozygous and 14% are homozygous for the H allele. Since the GYS1 H allele appears to be dominant we predict that approximately 7% of all Quarter Horses and Paint horses, 36% of all Belgians and 56% of all Percherons are actually genetically susceptible to PSSM.

TABLE 2
GYS1 Genotype Frequencies in Random Sample
Populations of Different Breeds

| | Quarter | Paint | | | Thorough- |
|---|---|---|---|---|---|
| Genotype | Horses | Horses | Belgian | Percheron | bred |
| R/R | 313 (93%) | 180 (92%) | 20 (61%) | 22 (44%) | 96 (100%) |
| R/H | 21 (6%) | 14 (7%) | 13 (26%) | 21 (42%) | 0 (0%) |
| H/H | 1 (<1%) | 1 (<1%) | 5 (10%) | 7 (14%) | 0 (0%) |

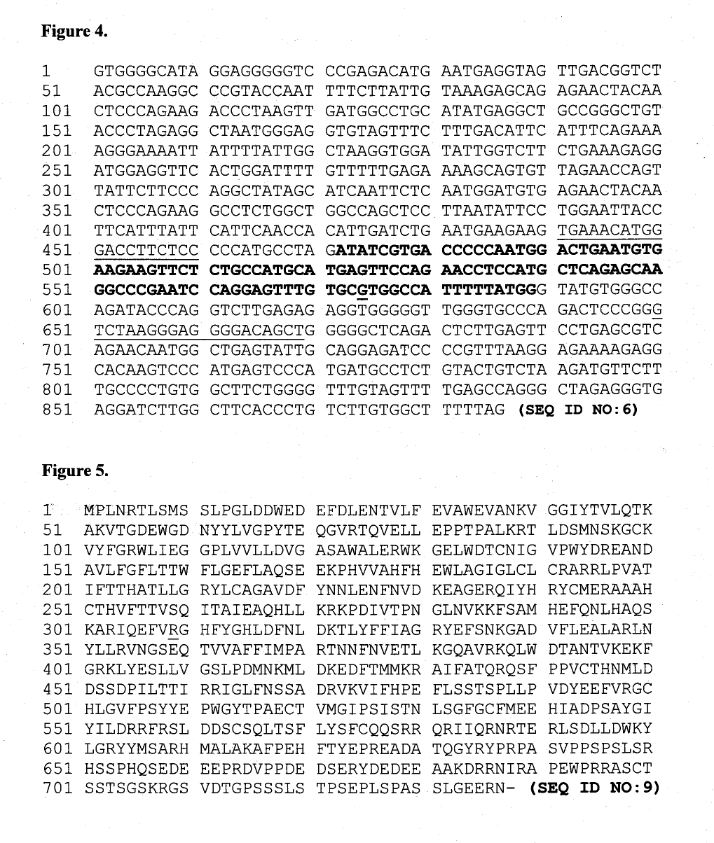

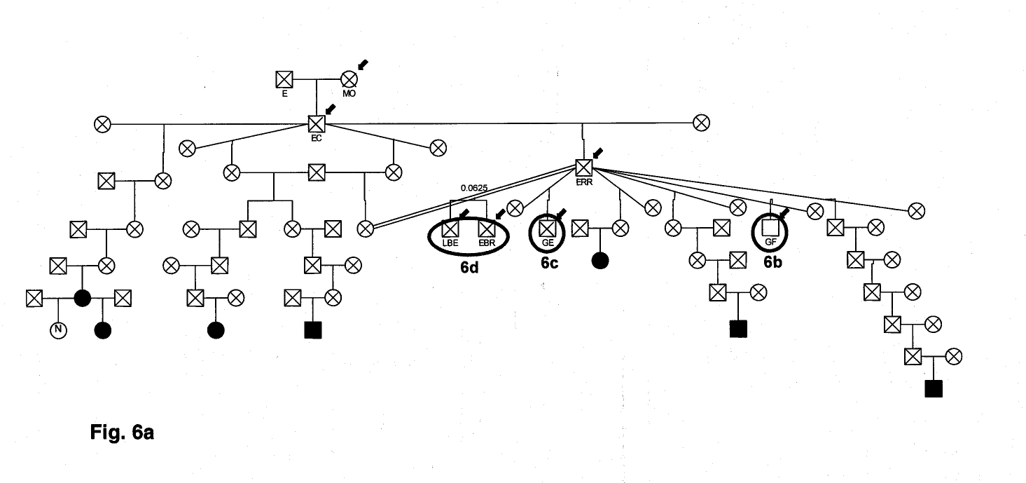

The nearly complete DNA sequence of the horse GYS1 gene (Horse GYS1 Intron 5, Exon 6, and Intron 6; FIG. 4) was assembled from sequences deposited into the NCBI trace sequence archive by the Broad Institute sequencing center during their recent equine whole genome shotgun sequencing project (SEQ ID NO:6). Introns and exons of the horse GYS1 gene sequence were then predicted from the homologous GYS1 exon sequences of other mammals. Intron 5 in this sequence comprises bases 1-471. Exon 6 in this sequence is highlighted and comprises bases 472-589. Intron 6 in this sequence comprises bases 590-886. The G to A mutation in exon 6 that causes the R to H amino acid mutation at codon 309 is underlined and is at base 574.

Using the GYS1 sequence, PCR primers are developed that can amplify the PSSM GYS1 mutation. For example, a PCR primer pair that has been successfully and reliably used to amplify this region from isolated horse DNA samples lies in introns 5 and 6 and the sequence locations are also underlined (FIG. 4). These sequences are 5′-TGAAACATGGGACCTTCTCC-3′ (SEQ ID NO:7) and 5′-AGCTGTCCCCTCCCTTAGAC-3′ (SEQ ID NO:8). Many other primer pairs are also possible.

Using the above PCR primers to amplify the region, the genotype of any horse (G/G, GIA or A/A for the DNA sequence, and R/R, R/H, and H/H for the amino acid sequence) can be obtained. In this method the restriction enzyme HypCH4 V cuts the GYS1 H allele at the exon 6 site (base 574), as well as at an intronic site 100 bp distant present in both the R and H alleles that serves to monitor enzyme efficiency. The products are separated by agarose gel electrophoresis and visualized by ethidium bromide staining under ultraviolet light. Many other methods of detecting the G or A nucleotide at this position of the horse GYS1 sequence are possible.

DNA testing based on the present invention now provides veterinarians and veterinary pathologists with a means to more accurately determine if a horse with clinical signs of PSSM has the heritable and most common form of disease that can be specifically attributed to this GYS1 gene mutation. All that is needed are a tissue sample containing the individual's DNA (typically hair root or blood) and appropriate PCR and sequence analysis technology to detect the G to A single nucleotide change. Such PCR primers are based in exon 6 and its flanking intron sequences as depicted in FIG. 2, sequences nearby this region depicted in FIG. 1, or in other DNA sequence from introns of this gene.

Also, DNA testing provides owners and breeders with a means to determine if any horse can be expected to produce offspring with this form of PSSM. An H/H horse would produce an affected foal 100% of the time, while an H/R horse would produce an affected foal 50% of the time when mated to an R/R horse. Mating of H/H and H/R horses would produce an affected foal 75% of the time. Breeding programs could incorporate this information in the selection of parents that could eventually reduce and even eliminate this form of PSSM in their herds.

### EXAMPLE 2

### Glycogen Synthase (GYS1) Mutation Causes a Novel Skeletal Muscle Glycogenosis

Horses have been domesticated for thousands of years, and modem breeds have been established by selection for a variety of athletic, physical and behavioral traits. In horses, as in other animal species, line breeding to propagate desirable traits has coincidently also propagated deleterious traits. One such undesirable trait is Polysaccharide Storage Myopathy (PSSM), a debilitating and potentially life-threatening glycogen storage disease that occurs in several genetically diverse breeds of horses. Clinical findings consistent with PSSM were first reported in the early 1900s in working Draft horses that developed exertional rhabdomyolysis when returning to work after several days of rest. However, PSSM was not recognized as a skeletal muscle glycogenosis until 1992. Today, as many as 36% of Draft horses and 10% of Quarter Horses have PSSM. The phenotypic expression of PSSM ranges from muscle atrophy and progressive weakness in Draft horse breeds to muscle soreness and gait abnormalities in Warmblood breeds, and acute exertional rhabdomyolysis in Quarter Horses. The severity of clinical signs in PSSM ranges from muscle cramping and stretching out to severe muscle pain and myoglobinuria and occasionally the complete inability to rise. Although PSSM is clinically well-recognized and common, its metabolic basis has remained a mystery for over a hundred years.

All horses with PSSM accumulate excess glycogen as well as abnormal amylase resistant polysaccharide in type 2A and type 2B skeletal muscle fibers. The disease has been best characterized in Quarter Horses, where a familial relationship, enhanced insulin sensitivity and blood glucose uptake, and myofiber energy deficit with sub-maximal exercise have been demonstrated. Although glycogenolytic and glycolytic defects are the most common cause of glycogenoses in humans, PSSM horses have normal glycogenolytic and glycolytic enzyme activities and are able to utilize glycogen and produce lactate with anaerobic exercise. Thus, PSSM appears to be a unique animal model of abnormal muscle glycogen metabolism that may provide insight into myocyte carbohydrate metabolism, and the genetic basis of uncharacterized glycogenoses in humans. The complexity of potentially defective pathways including insulin signaling, skeletal muscle glucose uptake and glycogen metabolism dictated that a positional cloning approach should be utilized to identify positional candidate genes.

Results

Genome-Wide Association Analysis

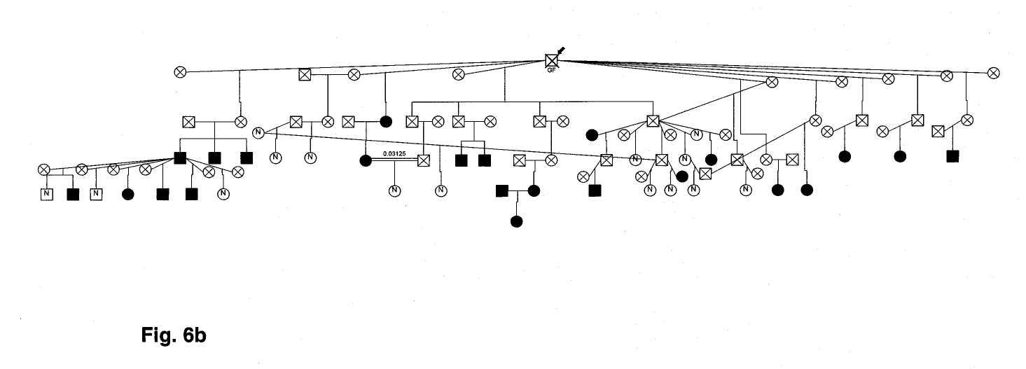

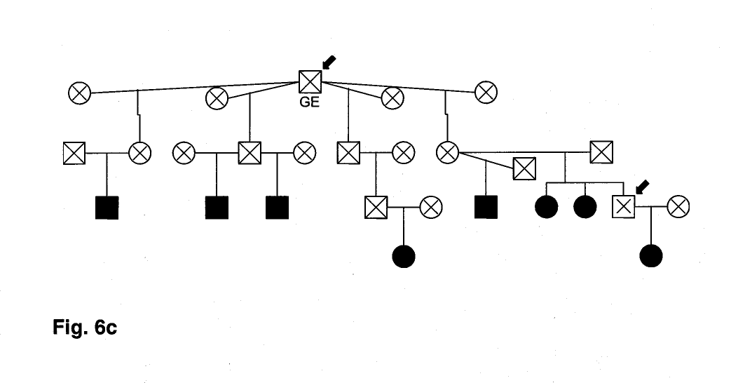

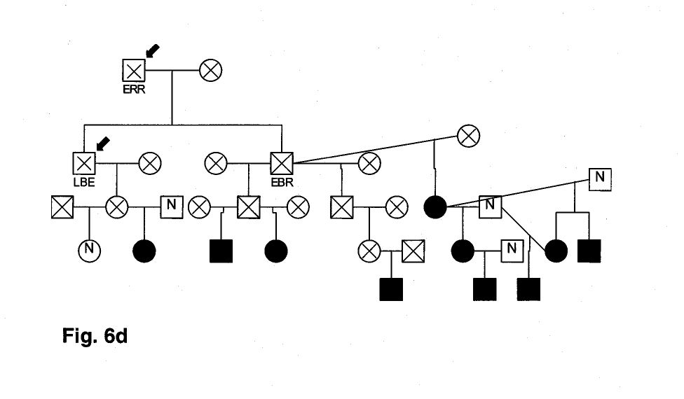

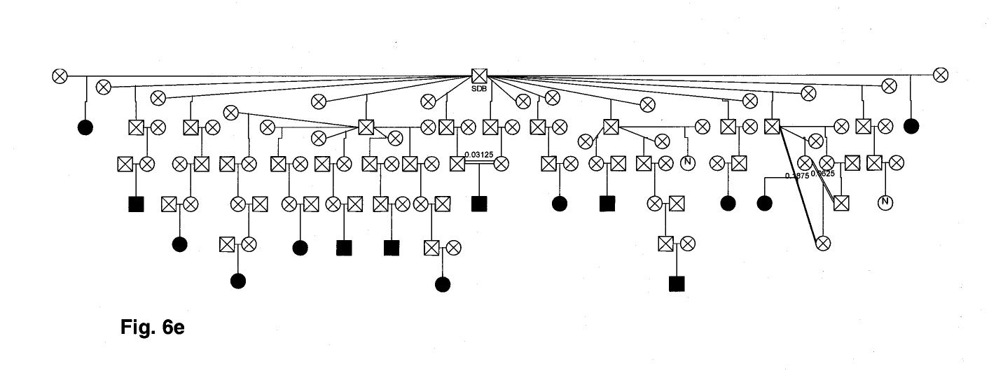

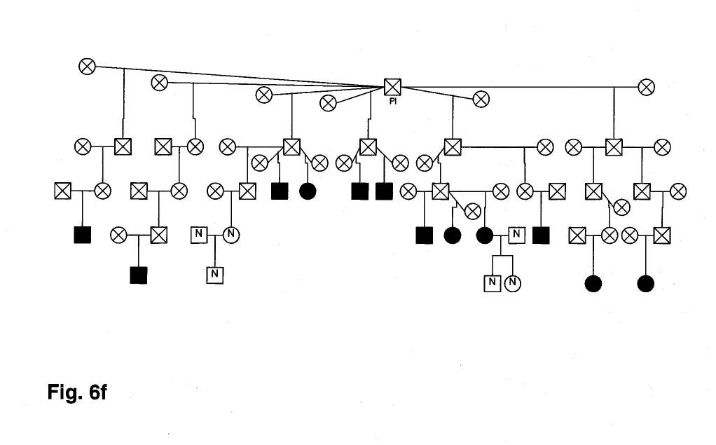

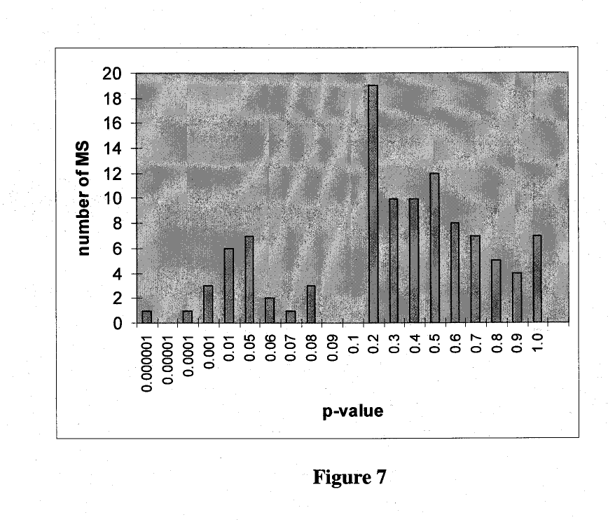

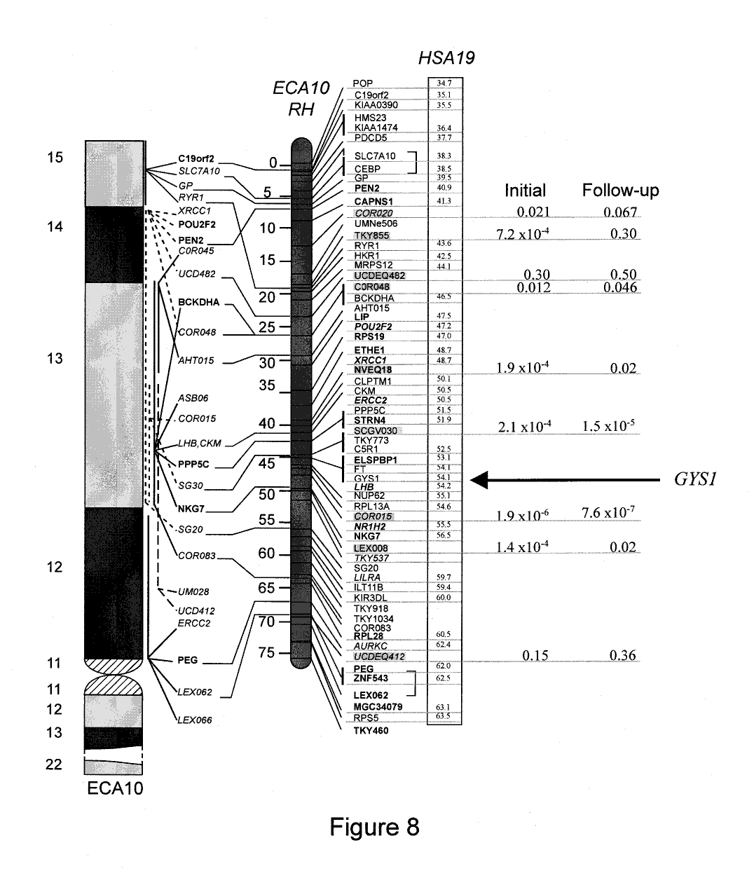

One hundred and five microsatellite (MS) markers distributed on all 31 equine autosomes were genotyped on an initial population of 48 related PSSM and 48 unrelated control Quarter Horses. The 48 related PSSM Quarter Horse cases were selected from an extended pedigree and all traced back to a single common ancestor within 7 generations, thus increasing the extent of the shared chromosomal segment surrounding the mutation and improving the power to detect association (see FIGS. 6a-6f for population structure). Chi square tests for association gave a range of p values from 1.9×10⁻⁵ to 0.97 for the comparison of allele frequencies between the PSSM and control groups (FIG. 7). Two markers on two different chromosomes had p-values <0.001. The NVEQ018 marker on ECA10p gave a p value of 1.9×10⁻⁵, and its allele distribution showed that 23/48 of the affected horses possessed an allele that was virtually absent (2/48) in the controls (FIG. 8).

Eight additional ECA10p MS were then genotyped to define an approximately 10 Mb interval that was highly associated with PSSM (p=2.1×10⁻⁴ to 1.9×10⁻⁷) (FIG. 8). To rule out spurious association due to the relatedness of the affected and control cohorts and thus dependence of the observations in the chi-square analysis, and to narrow the candidate chromosomal region, a follow-up population of 52 PSSM and 44 control Quarter Horses was genotyped. PSSM cases in the follow-up cohort were chosen on the basis of not sharing a common ancestor with the initial case cohort within 5 generations. The two markers with p values <2.0×10⁻⁵ in this follow-up population (COR015 and SCGV030) spanned an approximately 3 Mb region highly associated with PSSM (FIG. 8). The p values for these two markers in the combined population of 100 cases and 92 controls were 1.0×10⁻⁴ and 2.0×10⁻⁸.

The horse-human comparative map indicates that the region of ECA10p encompassed by COR015 and SCGV030 is homologous to human chromosome 19 (HSA19), from Mb position 51.9 to 54.6. Of the positional candidates in this region of HSA19, only the GYS1 gene at 54.1 Mb is known to play a direct role in carbohydrate metabolism. GYS1 encodes the skeletal muscle isoform of glycogen synthase (GS) that is expressed in skeletal muscle and non-hepatic tissues. GS catalyzes the addition of single glucosyl residues from UDP-glucose onto a glycogen polymer using an α1→4 glycosidic linkage and is the rate-limiting step in glycogen synthesis.

Identification of a Candidate Causal Mutation

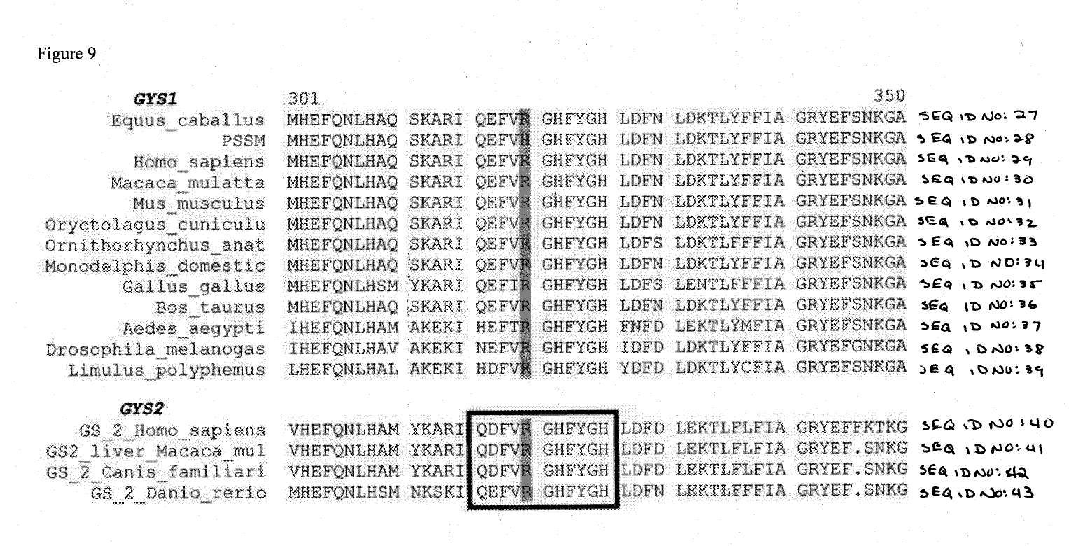

The GYS1 protein coding, as well as 5′- and 3′- untranslated sequences from a PSSM and a control Quarter Horse, were PCR amplified and sequenced from skeletal muscle cDNA or genomic DNA. The PSSM horse sequenced was selected because it was homozygous at both the Cor015 and SCGV030 loci and was likely to be homozygous for a potential mutation; the control horse was randomly selected from the controls used for the genome scan. A single polymorphism was identified, in which a G to A base substitution changes the normal arginine (R) at codon 309 (CGT) to a histidine (H) (CAT) in PSSM affected Quarter Horses. Sequence analysis of GYS1 exon 6 from genomic DNA was performed in 6 additional severely affected and 6 control horses. Two of the PSSM affected horses were homozygous and four were heterozygous for the histidine mutation while the 6 control horses were all homozygous for the normal arginine allele. Multiple alignment of the amino acid sequence in this region demonstrated that arginine 309, as well as the surrounding amino acids (305-QEFVRGHFYGH-314, SEQ ID NO:10), are highly conserved in both the GYS1 (muscle) and GYS2 (liver) forms of GS (FIG. 9).

Glycogen Synthase Activity in PSSM

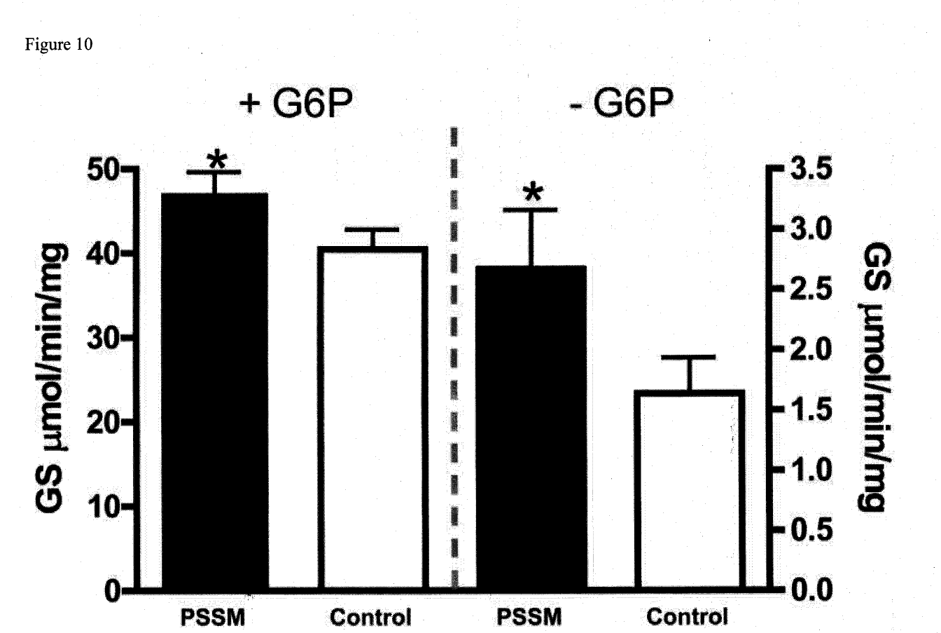

Glycogen synthase assays on muscle homogenates were performed. The mean GS activity was higher in PSSM than control muscle in both the presence and absence of the allosteric activator glucose 6-phosphate (G6P) (p=0.03 and 0.04, respectively) (FIG. 10). The −/+ G6P ratio was not significantly different between PSSM and control horses (p=0.09; mean=0.06 and 0.03 in PSSM and control respectively). The low −/+ G6P ratio suggests that the GS present in the skeletal muscle homogenates was highly phosphorylated.

GYS1 Allele Frequency in Quarter Horses

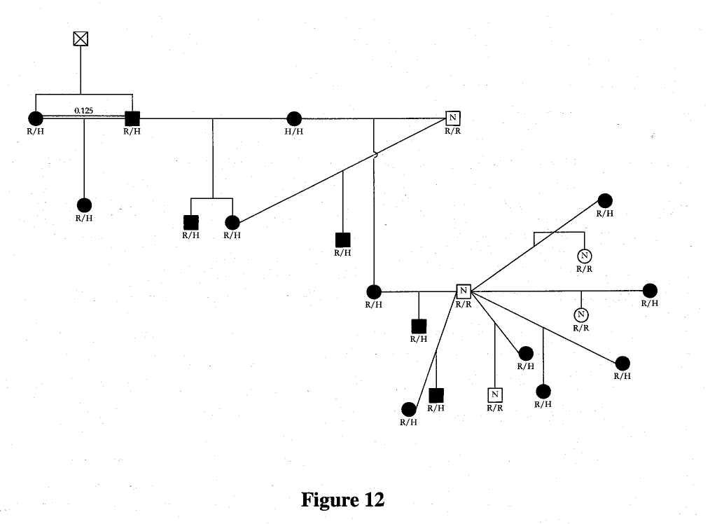

Ninety-nine PSSM Quarter Horses used in the whole genome association analysis were genotyped for the Arg309His mutation; 72 were heterozygous and 5 were homozygous for the H allele. Surprisingly, 22 PSSM horses were homozygous for the normal R allele. Eighty eight of the 92 control Quarter Horses were homozygous normal and 4 were heterozygous for the H allele. Based on these genotypes, penetrances of the H/H and R/H genotype are 1.0 and 0.95, respectively. However, a gluteal muscle biopsy is not a 100% sensitive diagnostic test, and the 4 control horses that were heterozygous for the H allele may have been false negatives and represent phenotypic error. Segregation of the H allele with PSSM was confirmed in a resource pedigree (FIG. 12).

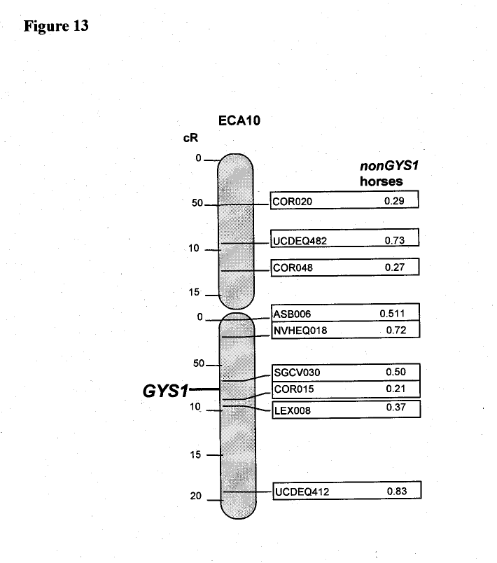

The population of PSSM horses without the GYS1 H allele suggests either that the G to A substitution is not the causative mutation or that the PSSM affected horses without the G to A substitution are phenocopies. Sequencing from 480 bp upstream of the predicted promoter region through exon 1, the entire coding sequence and the 3′ UTR (301 bp) did not detect any additional sequence differences between affected and control horses. Furthermore, association analysis using the same ECA10p MS markers excluded this chromosomal locus and GYS1 as the cause of abnormal polysaccharide accumulation in GYS1 R/R PSSM Quarter Horses (FIG. 13) and strongly suggests a second non-GYS1 glycogenosis exists that also produces exertional rhabdomyolysis and skeletal muscle polysaccharide accumulation in Quarter Horses.

GYS1 Mutation in Diverse Horse Breeds

Despite similarities in histopathologic findings in different breeds, there is variability in clinical findings in PSSM across breeds. 750 horses from diverse breeds diagnosed with PSSM on the basis of abnormal polysaccharide in skeletal muscle were genotyped for the GYS1 mutation. The His309 allele was found in either heterozygous or homozygous form in 356 horses from 15 different breeds including Quarter Horses, Paint horses, Appaloosa horses, 5 Draft horse breeds, 3 Warmblood breeds, the Morgan, Mustang, Rocky Mountain Horse breeds, as well as mixed breed horses and Warmblood horses of unspecified breed.

Haplotype Analysis and Allele Age

If the G to A GYS1 mutation is identical by descent in multiple horse breeds, it suggests that it was present in the domestic horse population before the establishment of the diverse breeds known today, and it has been passed from established breeds to newer breeds by admixture at the time of new breed creation. 767 horses from the breeds containing the Arg309His mutation were genotyped for 45 SNP markers in the 2 Mb region surrounding GYS1. Missing genotypes and haplotype phase were inferred using fastPHASE 1.2, and minimally conserved haplotype was determined using Haploview 4.0RC2. All chromosomes with the GYS1 A allele had a single conserved haplotype (FIG. 11). This conserved haplotype suggests that the A allele is identical by descent in all horse breeds. The conserved haplotype was the shortest in Belgian draft horses (357 Kb), consistent with the earlier origin of the Belgian breed and more decay of LD around the mutation. Quarter Horses, Paint horses and Appaloosas shared a larger conserved segment (618 Kb) likely due to the more recent origin of these breeds.

The mean allele age of the GYS1 mutation, estimated with DMLE+2.0, was 159 generations, median allele age was 163 generations, and mode was 153 generations. The range of generations with significant frequencies (p<0.05) was 140 to 189, and 90% of iterations fell between 144 and 178 generations. With the mean generational interval in horses estimated to be approximately 8 years, we conclude that this mutation likely originated between 1200 to 1500 years ago, prior to the separation of the modem breeds known today.

Discussion

Our results are consistent with the hypothesis that the PSSM phenotype in horses is due to a dominant gain of function mutation in the GYS1 gene that results in enhanced activity, and/or poor regulation of the mutant His309 enzyme. Support for a gain of function in GS resulting in a glycogenosis in mammals comes from mice over-expressing a rabbit skeletal muscle GS that is not inactivated by protein kinase catalyzed phosphorylation. Similar to PSSM horses, these mice accumulate excessive abnormal polysaccharide in the cytoplasm of skeletal myocytes which disrupts normal myofibrillar ultrastructure. The iodine absorption spectra of the abnormal polysaccharide in both GS over-expressing mice and PSSM horses is shifted to higher wavelengths, consistent with decreased branched structure of glycogen. Lastly, when exercised to exhaustion the mice were able to utilize skeletal muscle glycogen and had an exaggerated increase in blood lactate, suggesting an increased rate of anaerobic glycogenolysis and glycolysis similar to PSSM horses.

The action of GS is the rate-limiting step in glycogen synthesis; therefore its enzymatic activity is tightly controlled. GS activity is not extensively regulated through GYS1 gene expression in mammals because of the need to rapidly change from glycogen synthesis to degradation. Gain of function mutations are most commonly caused by increased expression of the mutant protein due to mutation in the promoter region of the gene. However, the GYS1 mutation identified in PSSM affected horses occurred in the coding sequence of exon 6, altering codon 309, and sequencing of the promoter and the 5′ UTR of the GS from PSSM affected horses did not demonstrate any sequence polymorphism. Furthermore, overexpression of GS in mammalian in vitro and in vivo systems, or in yeast systems, fails to result in glycogen accumulation, due to phosphorylation and inactivation of the overexpressed GS enzyme. Thus it is more likely that the glycogen accumulation in PSSM horse muscle is due to the Arg309His mutation and results from enhanced activity and/or poor regulation of the mutant enzyme.

The interaction between allosteric activation and covalent modification of GS by protein kinases may be best described by a three state model. In this model, phosphorylated GS in the absence of G6P is in a low activity state (state I) and dephosphorylated GS in the absence of G6P is in an intermediate activity state (state II). Phosphorylated enzyme (state I) requires near saturation with G6P to reach a high activity state (state III), whereas dephosphorylated GS (state II) is converted to a high activity state (state III) by relatively low concentrations of G6P. The increased activity of GS both in the presence and absence of G6P suggests that the Arg309His enzyme may be constitutively active in PSSM horses. However, data obtained from muscle homogenates make it difficult to determine if the increased GS activity is due to higher inherent enzyme activity, decreased response to phosphorylation, or altered sensitivity to allosteric activation by G6P.

The Arg309His mutation in PSSM horses occurs in a highly conserved region of GS. The importance of this residue and the highly conserved surrounding region has been demonstrated in both glycosyltransferase-3 (GT-3, mammalian and yeast) and glycosyltransferase-5 (GT-5, bacterial) families of GS. Mutation of the homologous residue in E. coli (Lys277) results in enzyme inactivation, while mutation of the adjacent residue in yeast (Gly310) results in enhanced activity. Despite clear evidence that this region of the GS enzyme is important to overall activity, the mechanism by which an Arg309His mutation results in alteration of GS function in eukaryotes is unclear since the crystal structure has not been solved for any member of the GT-3 family and this region has not been well studied by mutagenesis in GT-3 GS. However, residues 305-314 are not among the homologous highly-conserved mammalian GT-3 residues implicated in glucosyl donor substrate binding or catalytic activity.

Constitutively active GS in PSSM could result from altered regulation by phosphorylation or enhanced sensitivity to allosteric activation by G6P. Residue 309 is distant to the known carboxyl and amino terminal phosphorylation sites in GT-3 GS, making it unlikely that it directly inhibits phosphorylation; however this does not rule out the mutation from causing resistance to negative regulation due to inappropriate response to phosphorylation, as observed in a Gly298Asp yeast mutant (Gly298 in yeast GS is analogous to Gly310 in mammalian GS). Altered enzyme activity in response to both negative regulation by phosphorylation and positive regulation by G6P could also potentially be due to the interaction of residues 305-314 with the C-terminal Arginine-rich cluster (R578A/R579A/R581A and R585A/R587A/R590A), a critical region of the GS enzyme which may act as a molecular switch allowing GS to toggle between states I, II and III. The Arg309His mutation could therefore potentially interfere with the metabolic switch resulting in GS being continuously in an active form. Definitive evidence for this mutation altering the regulation of GS would necessitate further in vitro study of Arg309His enzyme kinetics.

GYS1 His309 allele frequencies range from 0.035 to 0.350 in different breeds, which is surprisingly high considering that it is associated with a dominantly inherited neuromuscular disease. The high prevalence of this mutation in modern horse breeds may be due to the inadvertent breeding of affected individuals that were not recognized as having a deleterious genetic disease. Clinical signs of PSSM can readily be ameliorated if horses receive consistent daily exercise and limited starch intake. Thus, over most of the last 1200 years, when horses were used on a daily basis for work and transportation, and feed could be scarce, the GYS1 mutation might not have conferred a selectional disadvantage. However, in recent times horses have increasingly become companions that are exercised less frequently, housed in small spaces and fed high starch concentrates. In this management scenario, horses with the GYS1 His309 allele are more likely to develop muscle pain associated with PSSM. It is conceivable that the GYS1 mutation may have previously conferred a selectional advantage due to promotion of an increased skeletal muscle glycogen content when feed sources were scare.

To date, no other naturally occurring gain of function mutations in GYS1 have been shown to cause a glycogen storage disease in any mammalian species. The related GYS2 gene, expressed in liver, has several known mutations that reduce GS enzyme activity and hepatic glycogen content, leading to fasting hypoglycemia. Recently, a mutation in the human GYS1 gene resulting in a stop codon in exon 11, a recessively inherited cardiac and skeletal muscle glycogen deficiency, hypertrophic cardiomyopathy, and reduced exercise tolerance, has been described. However, the dominantly inherited PSSM GYS1 mutation appears to result in an increased GS activity in skeletal muscle, making it a new form of glycogen storage disease. It may be useful to examine GYS1 as a candidate gene for the significant fraction of human glycogen storage myopathies with as yet undetermined molecular bases.

Horses are particularly useful for the study of metabolic myopathies as they are typically superb athletes bred for speed or endurance, and disturbances in any pathway involving energy metabolism or muscle function are readily apparent since they are not provided the option of being sedentary. Further understanding of the mechanism responsible for altered glycogen and glucose metabolism in the GYS1 and non-GYS1 forms of equine PSSM may also provide new insights into normal and abnormal regulation of muscle glycogen metabolism and the development of exertional rhabdomyolysis.

Methods

Genome-Wide Association Analysis

PSSM positive cases were selected from biopsy submissions to the Neuromuscular Disease Laboratory (NDL) at the University of Minnesota as well as from a population of Quarter Horses identified in a previous prevalence study (McCue et al., J Vet Intern Med. 2006; 20:710). Case criteria for a positive diagnosis of PSSM included: 1) the presence of (PAS)-positive, inclusions of polysaccharide in gluteal or semimembranosus muscle fibers that are typically amylase resistant, and 2) 1.5 to 2 times normal muscle glycogen levels, and/or 3) a clinical history of exertional rhabdomyolysis. Control Quarter Horses were also selected from horses biopsied in the prevalence study (McCue et al., J Vet Intern Med. 2006; 20:710). Control criteria included: 1) a normal gluteal muscle biopsy that verified the absence of abnormal PAS staining, 2) normal glycogen levels, and 3) no history of neuromuscular disease.

All horses (cases and controls) with registry information were tracked to the original breed founders (10-20 generations) and pedigrees were analyzed to determine the level of relatedness among individuals. Extended family groupings consisting of several individuals tracing back to a common ancestor within a short (2-8) generational interval were identified.

For the initial genome wide association study the goal was to maximize the length of linkage disequilibrium (LD) and minimize locus and genotypic heterogeneity in the study population, thus increasing the power to detect association. This was accomplished by selecting 48 PSSM affected horses from the extended families identified in the pedigree analysis. Because the PSSM horses were closely related LD was maximized; minimal recombination would occur between microsatellite (MS) markers flanking the founder PSSM locus. Forty eight PSSM cases were selected from 3 extended families identified in the pedigree analysis (FIG. 6). To avoid false inflation of association due to sampling of closely related individuals, we did not include individuals that shared more genetic material than second cousins. Control Quarter Horses were not related to the case cohort and were not more closely related than third cousins.

Sample sizes for the initial genome scan were estimated using a genetic power calculator using the following data. Disease prevalence was set at 0.06; high risk allele frequency (A) at 0.04 (expected frequency of the PSSM mutation in the population if it is inherited as a dominant disease trait based on a conservative estimate of 6% prevalence in the Quarter Horse population); Aa and AA genotypic relative risks were set at 90 and 95 respectively. To estimate D′ and marker high risk allele frequency for power calculations, previously generated genotype data from forty-two markers across 7 different chromosomes from 96 PSSM and control Quarter Horses was analyzed. The mean D′ value for marker pairs with significant allelic association was 0.47 and the mean allele frequency across all alleles of all markers was 0.15. Therefore, D′ was estimated at 0.47; and high risk allele frequency for the marker allele was set at 0.15. Based on these criteria 41.85 cases and an equal number of controls were required for 85% power with a 5% type I error rate (p=0.05), which is in agreement with other published calculations (Chapman et al. Am J Hum Genet. 1998;63:1872-1885).

The initial 105 MS markers genotyped were selected from a group of markers known to be highly heterozygous in several breeds and were distributed across all 31 autosomes. DNA samples from case and control cohorts were genotyped for each MS marker using a Beckman CEQ8000 automated DNA fragment analyzer. The genotypes for each of the MS were used to compare allele frequencies in the case and control cohorts. The number of alleles of each size detected in each cohort were counted (n horses×2 alleles/horse=2n total alleles), and allele frequency in each cohort determined. Minor alleles, defined as any allele with a frequency less than 10% in the entire study population (cases and controls), were summed for statistical analysis. Case and control cohort allele frequencies were compared with Pearson's χ² test of independence to determine if there was a significant difference between the allele distribution in the two cohorts.

Identification of a Candidate Causal Mutation

The entire equine GYS1 DNA sequence was assembled from the NCBI trace files derived from the Broad Institute equine whole genome 7× shotgun sequence. The predicted equine GYS1 sequences were used to design primers for PCR amplification and sequencing. The 5′ untranslated region (UTR) and exon 1 were PCR amplified from genomic DNA with two primer pairs (5 ′ACA GAG CTG AGG GCC AAT C (SEQ ID NO: 11)/ 5′ CCG GCT CCC TGT TAC TCA AG (SEQ ID NO:12) and 5′AAA GAT CTC TGT GCT CCC TCA (SEQ ID NO:13)/ 5′GGC CCT GCA GTG TAA CCT T (SEQ ID NO:14)). Exon 16 and the 3′ UTR were PCR amplified from genomic DNA with two primer pairs (5′GGG ATG TGG CTC AGA GAC TG (SEQ ID NO:15)/ 5′TAG CGG TAG CCC TGG GTC TG (SEQ ID NO:16) and 5′CTC CGC TCA CTC TGC AGT C (SEQ ID NO:17)/ 5′ACC TCT GGC ACC ACA GTA GG (SEQ ID NO:18)). Exons 2-15 were amplified from skeletal muscle cDNA prepared from an affected and control horse. cDNA was amplified using three overlapping primer pairs (5′GCA CTT TGT CCA TGT CCT CA (SEQ ID NO:19)/ 5′TTT ATG GGC ACC TGG ACT TC (SEQ ID NO:20), 5′GAG GCT CAG CAC CTA CTC AAG (SEQ ID NO:21)/ 5′CAT CCC CAG TAT CTC CAC CA (SEQ ID NO:22), and 5′TAT GAG CCT TGG GGC TAC AC (SEQ ID NO:23)/ 5′CGA TGA AGA CAG TGA GCG CT (SEQ ID NO:24)).

Glycogen Synthase Activity in PSSM

Glycogen synthase activity was measured in flash frozen gluteal muscle biopsy samples from 17 PSSM and 15 control Quarter Horses (11 PSSM and 12 controls without G6P). GS activity was assayed by measuring the incorporation of glucose from UDP-[U-¹⁴C] glucose into glycogen. 20 mg of frozen muscle was added to homogenate solution (50 mM Tris Acetate, 20 mM NaF, 2 mM EDTA, 1 mM mercaptoethanol, pH 7.8) at 2% wt./vol and homogenates were made using a Potter Elvehjem handheld glass homogenizer. Samples were centrifuged at 4° C. and 1000 g for 10 min and supernatant saved for GS assay. GS activity was assayed by measuring the incorporation of glucose from UDP-[U-¹⁴C] glucose into glycogen using 6.7 mg/ml de-ionized rabbit liver glycogen and 5 mM UDP-glucose (˜150 cpm/nmol) both with (maximal activity) and without 7.2 mM glucose-6-phosphate (G6P). ¹⁴C-labeled glycogen was spotted onto Whatman® no. 5 filter paper and washed in 66% ethanol for 20 mm to remove UDP-[U-¹⁴C] glucose. Filters were washed with acetone for 5 min and dried before counting in a liquid scintillation counter. A unit of activity was defined as the amount of enzyme that catalyzed the transfer of 1 μmol glucose from UDP-glucose to glycogen in one minute. Data is expressed as units of GS activity per mg of GS protein. The −/+ glucose-6-phophate ratio was calculated by dividing the activity of GS in the absence of G6P by the activity in the presence of 7.2 mM G6P. Significant differences in GS activity between PSSM and control horses were determined using an unpaired t-test. Significance level was set at p<0.05.

GYS1 Allele Frequency in Quarter Horses

The G to A base substitution identified in exon 6 was rapidly genotyped in PSSM and control horses using a restriction fragment length polymorphism (RFLP) assay. A 230 bp segment of DNA containing GYS1 exon 6 and the flanking intronic sequence was amplified by PCR (forward/reverse primer 5′TGA AAC ATG GGA CCT TCT CC (SEQ ID NO:25)/ 5′AGC TGT CCC CTC CCT TAG AC (SEQ ID NO:26)). In horses without the G to A polymorphism a single restriction site for the enzyme HpyCH4 V is present in this fragment; in horses with the G to A polymorphism a second restriction site is created. Restriction fragments were resolved using 3% agarose gel electrophoresis.

GYS1 Mutation in Diverse Horse Breeds

Records from muscle biopsy submissions to the University of Minnesota Neuromuscular Diagnostic Laboratory between January 1996 and January 2007 were reviewed. Submissions from clinical cases and from research horses were included. 812 PSSM horses from 35 different breeds were identified from the Neuromuscular Disease Laboratory database. 750 cases had whole blood or tissue that was available for DNA isolation and genotyping for the GYS1 mutation. Genomic DNA was isolated from whole blood samples using the Genomic DNA Purification Kit (Gentra, Minneapolis, Minn.) according to the manufacturer's protocol. When whole blood was not available, genomic DNA was isolated from muscle biopsies (frozen or formalized) using DNeasy® Tissue Extraction Kit (Qiagen®, Valencia, Calif.) according to the manufacturer's protocol. The GYS1 genotype of each horse was obtained using the restriction fragment length polymorphism assay described above.

Haplotype Analysis and Allele Age

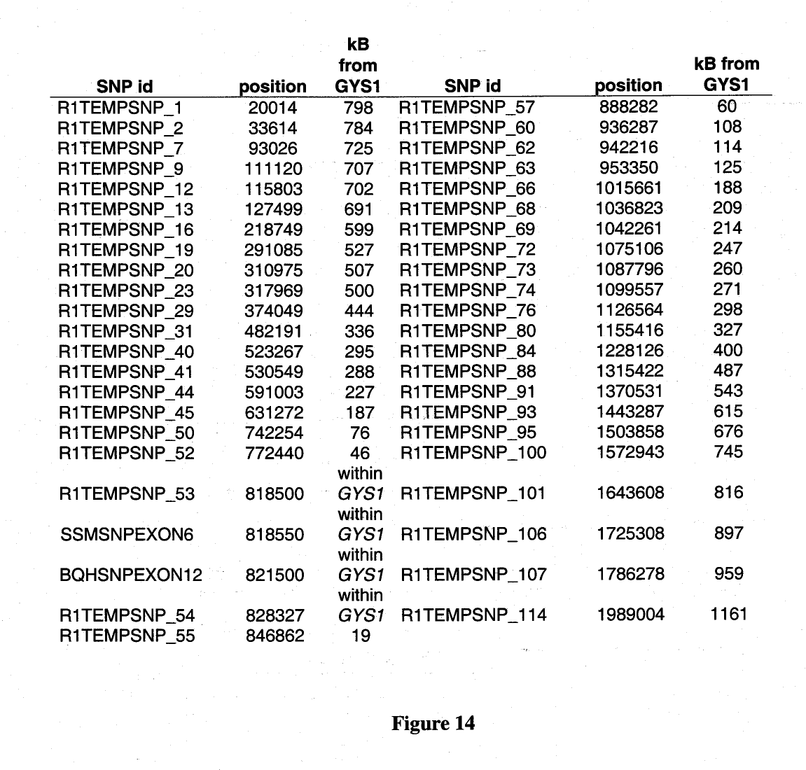

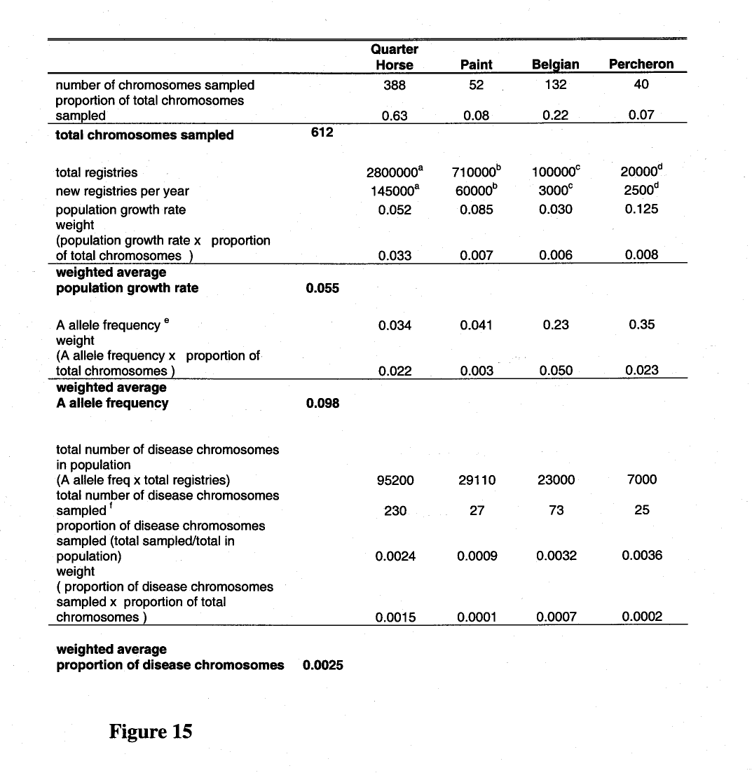

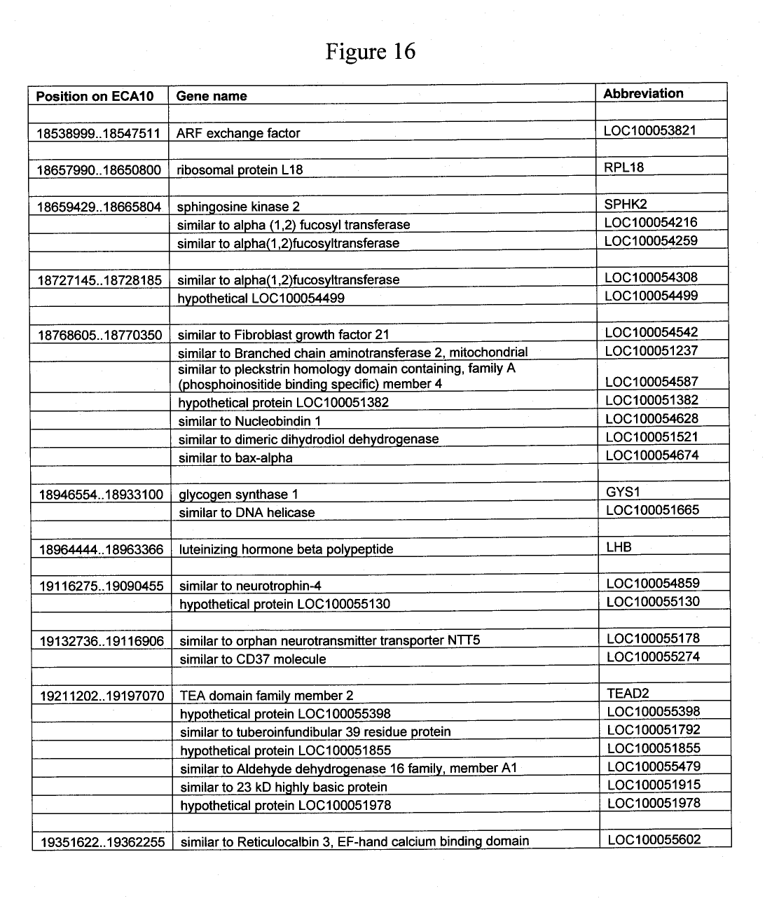

43 SNPs were selected for genotyping from a group of 100 well-characterized SNPs in the 2 Mb region surrounding GYS1. SNPs were genotyped using the Sequenom® platform. Primers were designed using SpectroDESIGNER software (on the world-wide-web at sequenom.com/Seq_genotyping.html). The 43 SNP loci (FIG. 14) were amplified in 4 multiplex PCR reactions with 1 to 28 primer pairs per reaction.

Missing genotypes and haplotype phase were inferred using fastPHASE 1.2 software, with the following options included in the algorithm: -H200 to increase the number of haplotypes sampled from the posterior distribution from a particular random start of the EM algorithm to 200 to minimize individual error; -KU30 -KL5 -Ki2 to increase the number of clusters considered (minimum of 5 to maximum of 30 at interval of 2) due to the large number of individuals and the complexity of the dataset due to large number of subpopulations; -u to incorporate subpopulation labels to deal with any deviation from Hardy Weinberg equilibrium (HWE) due to subpopulation differences in allele and haplotypic frequencies; -e to scan for genotype errors across all SNPs. Horses were grouped into subpopulations by breed. Minimally conserved haplotype was determined using Haploview 4.0 CR2. Data from individuals for a given breed were imported into Haploview as phased haplotypes. Markers were checked with default parameters of HWE p-value cut-off of p=0.001 and a minimum minor allele frequency of 0.001. The GYS1 exon 6 G/A SNP (responsible for the Arg309His mutation) was manually included, despite not achieving HWE cut-off, which was expected based on the cohort selection. Haplotype blocks were manually extended across SNPs on either side of the exon 6 SNP to determine the conserved haplotype around the GYS1 A allele. Haplotypes which occurred in less than 1% of the cohort were assumed to likely be due to genotyping error and were not considered.

GYS1 allele age was estimated using the DMLE+2.0 software. Due to the need for an accurate estimation of disease prevalence in the population, only haplotype data from Quarter Horses, Paint Horses, Belgian and Percheron horses were included in the allele age estimation, because these breeds had GYS1 allele frequency data available from genotyping random hair root samples. Population growth rates for each of these breeds were determined using numbers for total registered population and new registries per year obtained from the breed registries. Weighted averages for allele frequency, population growth rate and proportion of disease chromosomes sampled were calculated based on the proportion total chromosomes accounted for by each breed (FIG. 15). Weighted averages were used for parameter estimation in the DMLE+input. Iterations were performed over ancestral states and mutation age but not mutation location or allele frequency. Mean allele age was calculated from the histogram data for the posterior distribution of the time since the original mutation.

All publications are incorporated by reference herein, as though individually incorporated by reference. The invention has been described with reference to various specific and preferred embodiments and techniques. However, it should be understood that many variations and modifications may be made while remaining within the scope of the invention.

What is claimed is:

1. A method for detecting the presence of Polysaccharide Storage Myopathy (PSSM) in a horse, comprising the step of detecting in a nucleic acid sample from a horse a PSSM associated allele that is in linkage disequilibrium with glycogen synthase enzyme 1 (GYS1), wherein the presence of an allele with GYS1 is indicative of the horse being predisposed to or has PSSM.

2. The method of claim 1, wherein prior to or in conjunction with detection, the nucleic acid sample is subject to an amplification step.

3. The method of claim 1, wherein the detecting step is by a) allele specific hybridization; b) size analysis; c) sequencing; d) hybridization; e) 5′ nuclease digestion; f) single-stranded conformation polymorphism; g) primer specific extension; and/or h) oligonucleotide ligation assay.

4. The method of claim 3, wherein the size analysis is preceded by a restriction enzyme digestion.

5. The method according to claim 3, wherein at least one oligonucleotide probe is immobilized on a solid surface.

6. The method of claim 1, wherein the PSSM associated allele is located in a region of equine chromosome-10 within 500 kb of GYS1 exon 6.

7. A kit for determining the existence of or a susceptibility to developing Polysaccharide Storage Myopathy (PSSM) in a horse, the kit comprising a first primer oligonucleotide that hybridizes 5′ or 3′ to an allele in linkage disequilibrium with the exon 6 of glycogen synthase enzyme 1 (GYS1).

8. The kit of claim 7, which additionally comprises a second primer oligonucleotide that hybridizes either 3′ or 5′ respectively to the allele, so that the allele can be amplified.

9. The kit of claim 8, wherein the first primer and the second primer hybridize to a region in the range of between about 50 and about 1000 base pairs.

10. The kit of claim 7, which additionally comprises a detection means.

11. The kit of claim 10, wherein the detection means is by a) allele specific hybridization; b) size analysis; c) sequencing; d) hybridization; e) 5′ nuclease digestion; f) single-stranded conformation polymorphism; g) primer specific extension; and/or h) oligonucleotide ligation assay.

12. The kit of claim 7, which additionally comprises an amplification means.
